(19) United States
(12) Patent Application Publication
(10) Pub. No.: US 2022/0333053 A1
(43) Pub. Date: Oct. 20, 2022

(54) DEVICE AND METHOD FOR GENETIC ANALYSIS OF PLANT MATERIALS IN REMOTE TESTING SITES

(71) Applicants: The Johns Hopkins University, Baltimore, MD (US); Pioneer Hi-Bred International, Inc., Johnston, IA (US)

(72) Inventors: Tza-Huei Wang, Germantown, MD (US); Dong Jin Shin, Baltimore, MD (US); Fan-En Chen, Baltimore, MD (US); Yue Yun, Johnston, IA (US); Arturo M. Escajeda, Oakland, CA (US); Ming X. Tan, San Ramon, CA (US); Justin Schares, Ames, IA (US); Blake Freml, Urbandale, IA (US)

(73) Assignees: The Johns Hopkins University, Baltimore, MD (US); Pioneer Hi-Bred International, Inc., Johnston, IA (US)

(21) Appl. No.: 17/641,071

(22) PCT Filed: Sep. 10, 2020
(86) PCT No.: PCT/US2020/050195

§ 371 Date: Mar. 7, 2022

Related U.S. Application Data
(60) Provisional application No. 62/898,224, filed on Sep. 10, 2019.

(51) Int. Cl. C12M 3/06; B01L 3/00; C12N 15/10; G01N 35/00

(52) U.S. Cl. CPC C12M 23/16; B01L 3/502; C12N 15/1017; G01N 35/0098; B01L 2200/16; B01L 2300/0681; B01L 2400/043; G01N 2001/4088

(57) ABSTRACT

Embodiments of the invention relate to devices for assaying a biomolecule from a plant sample including: a microfluidic cartridge for assaying a biomolecule from a plant sample, including: a top layer; and a bottom layer spaced apart from the top layer in a generally parallel orientation with respect to the top layer, the bottom layer defining a plurality of wells therein that protrude from a surface of the bottom layer; and a filter module for filtering the plant sample, including a filter body defining: an upper portion including an inlet structure forming an inlet channel; and a bottom portion configured to accept and secure a filter membrane. The filter body is configured to accept a microvolume aliquot of the plant sample, the bottom structure includes an outlet structure forming an outlet channel on an outlet side of the filter membrane, and at least one of the plurality of wells includes an assay reagent solution.

### CROSS-REFERENCE OF RELATED APPLICATION

This application claims priority to U.S. Provisional Application No. 62/898,224 filed on Sep. 10, 2019, the entire contents of which are hereby incorporated by reference.

### GOVERNMENT LICENSE RIGHTS

This invention was made with government support under R01A1117032 awarded by the National Institutes of Health. The government has certain rights in this invention.

### BACKGROUND

### 1. Technical Field

The field of the currently claimed embodiments of this invention relates to devices and methods for the genetic analysis of plant materials in remote testing sites.

### 2. Discussion of Related Art

The absence of a field-deployable solution to performing genetic analysis of plants leads to logistical challenges for plant trait screening in remote locations around the globe. The ability to identify genetic traits of plants directly at the site of sample acquisition confers the ability to make decisions more quickly and accurately. For example, monitoring biomarkers for traits related to disease susceptibility has an important function in monitoring the epidemiology of diseases and the evolutionary selection of traits. In another instance, detection and characterization of genetic markers in crops that are linked to traits of agronomic importance is an important task for the agroindustry. However, the current state of the art relies on the use of laboratory-bound techniques which preclude the testing of plant samples directly at the site of acquisition. In particular, current technology for nucleic acid extraction from plant sample, purification and analysis require the use of conventional laboratory equipment including centrifuges, heat blocks and thermal cyclers. Thus there remains a need for the development of devices and methods for the rapid and efficient genetic analysis of plant materials in remote testing sites sans the use of large or expensive traditional laboratory equipment.

### SUMMARY

An embodiment of the invention relates to a device for assaying a biomolecule from a plant sample including: a microfluidic cartridge for assaying a biomolecule from a plant sample, including: a top layer; and a bottom layer spaced apart from the top layer in a generally parallel orientation with respect to the top layer, the bottom layer defining a plurality of wells therein that protrude from a surface of the bottom layer; and a filter module for filtering the plant sample, including a filter body defining: an upper portion including an inlet structure forming an inlet channel; and a bottom portion configured to accept and secure a filter membrane. In such an embodiment, the filter body is configured to accept a microvolume aliquot of the plant sample, the bottom structure includes an outlet structure forming an outlet channel on an outlet side of the filter membrane, and at least one of the plurality of wells includes an assay reagent solution.

An embodiment of the invention relates to a method of detecting a biomolecule in a plant sample. The method includes the steps of: preparing a lysate including the plant sample by contacting the plant sample with a lysis buffer; filtering a microvolume aliquot of the lysate using a filter module; loading the filtered plant sample into a sample well of a microfluidic cartridge; amplifying the biomolecule; and detecting the biomolecule. The step of preparing the lysate and the filtering the microvolume aliquot of the lysate are done at an ambient temperature.

An embodiment of the invention relates to a filter module for filtering a plant sample, including a fluid-tight filter body defining: an upper portion including an inlet structure forming an inlet channel; and a bottom portion configured to accept and secure a filter membrane. The fluid-tight filter body is configured to accept a microvolume aliquot of the plant sample. The bottom portion includes an outlet structure forming an outlet channel on an outlet side of the filter membrane. Also, the outlet structure is configured to mechanically connect the bottom portion with a microfluidic cartridge.

An embodiment of the invention relates to a device for assaying a nucleic acid sequence from a plant sample including: a microfluidic cartridge for assaying a nucleic acid sequence from a plant sample; and a filter module for filtering the plant sample. The microfluidic cartridge includes: a top layer forming an inlet; and a bottom layer spaced apart from the top layer in a generally parallel orientation with respect to the top layer, the bottom layer defining a plurality of wells therein that protrude from a surface of the bottom layer. The filter module for filtering the plant sample includes a fluid-tight filter body defining: an upper portion including an inlet structure forming an inlet channel; and a bottom portion configured to accept and secure a filter membrane. The fluid-tight filter body is configured to accept a microvolume aliquot of the plant sample. The bottom portion includes an outlet structure forming an outlet channel on an outlet side of the filter membrane. Also, the outlet structure is configured to mechanically connect the bottom portion with the inlet of the top layer of the microfluidic cartridge.

### BRIEF DESCRIPTION OF THE DRAWINGS

Further objectives and advantages will become apparent from a consideration of the description, drawings, and examples.

FIG. 1 is a schematic showing a general approach for using a device having an integrated filter module according to an embodiment of the device.

FIGS. 2A and 2B are schematics showing a device for assaying a biomolecule from a plant sample according to an embodiment of the invention.

FIGS. 3A and 3B are exploded top and bottom views, respectively, of the device of FIGS. 2A and 2B.

FIG. 3C is a side view of the device of FIGS. 2A and 2B.

FIG. 4 shows a series of images demonstrating how a top layer and a bottom layer are assembled to form a device for assaying a nucleic acid sequence from a plant sample according to an embodiment of the invention.

FIG. 5 is a schematic showing the use of wax and oil (top image) or wax 2101 to form a seal over various reagents deposited into wells of a device for assaying a nucleic acid sequence from a plant sample according to an embodiment of the invention.

FIG. 6A is an illustration of a filter module according to an embodiment of the invention.

FIG. 6B is an exploded view of a filter module according to an embodiment of the invention.

FIG. 6C is an illustration of a device for assaying a nucleic acid sequence from a plant sample according to an embodiment of the invention.

FIG. 7 is a schematic showing a method for genetic analysis of a plant specimen according to an embodiment of the invention.

FIG. 8 is a schematic showing a method for genetic analysis of a plant specimen according to an embodiment of the invention.

FIG. 9 is a table showing comparison between the use of a lyophilized lysis buffer versus a fresh lysis buffer according to an embodiment of the invention.

FIG. 10 is a collection of images and graphs showing the filtration-based removal of precipitants according to an embodiment of the invention.

FIG. 11A is an exploded view of a filter module assembly according to an embodiment of the invention.

FIG. 11B is a cross-sectional view of the filter module assembly from FIG. 11A.

FIG. 12A is a side view and perspective view of a filter module assembly according to an embodiment of the invention.

FIG. 12B is a cross-sectional view of the filter module assembly from FIG. 12A.

FIG. 13A is an illustration of a filter module assembly coupled to a microfluidic cartridge according to an embodiment of the invention.

FIG. 13B is a cross-sectional view of the filter module assembly coupled to the microfluidic cartridge of FIG. 13A.

FIG. 13C is a bottom view of the filter module assembly coupled to the microfluidic cartridge of FIG. 13A.

FIG. 13D is a perspective view of the filter module assembly coupled to the microfluidic cartridge of FIG. 13A.

FIG. 14 is a table disclosing the performance of filtration-based lysate preparation according to an embodiment of the invention.

FIG. 15 is a schematic showing use of a microfluidic cartridge to process a biomolecule according to an embodiment of the invention.

FIGS. 16A-16C are a series of charts showing results for manual versus robotic agitation of magnetic beads according to an embodiment of the invention.

FIG. 17 is a series of charts showing the overall sample to result time according to an embodiment of the invention.

FIG. 18 is a series of graphs showing the detection of hydrolysis probe markers using a PCR assay for maize samples MO17, SX19 and B73, using the complete laboratory-free workflow for plant lysate preparation, nucleic acid purification and analysis according to an embodiment of the invention.

FIG. 19 is a plot showing results of an allelic discrimination assay according to an embodiment of the invention.

FIG. 20 is a graph showing results of a biomarker quantification assay according to an embodiment of the invention.

FIG. 21 is an illustration of a device for assaying a nucleic acid sequence from a plant sample according to an embodiment of the invention.

FIGS. 22A-22E are schematics of a device for assaying a biomolecule from a plant sample according to an embodiment of the invention.

FIG. 23 is an exploded view of a device for assaying a biomolecule from a plant sample according to an embodiment of the invention.

FIG. 24 is a bottom perspective view of the device of FIG. 23.

FIG. 25 is a top view of the device of FIG. 23.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

As used throughout, the term “biomolecule” refers to one or more of a protein, a nucleic acid, a carbohydrate, or a lipid. In some embodiments, the term “biomolecule” refers to a protein, an amino acid sequence, or a nucleic acid sequence. In some embodiments, the biomolecule is obtained from a plant sample.

As used throughout, the term “microvolume” is intended to be a volume in the range from 1 microliter to 1000 microliters.

As used throughout, the term “anterior orientation” with respect to a second filter membrane refers to a conformation where the second filter membrane is disposed in a filter module and is between the upper portion and a first filter membrane.

As used throughout, the term “microfluidic device” refers to a device for accepting and processing a biomolecule from a sample. Non-limiting examples of a microfluidic device include a microfluidic cartridge, a magnetofluidic cartridge, a magnetofluidic platform, and a magnetofluidic device. In some embodiments the microfluidic device is disposable. In some embodiments the microfluidic device is preloaded with magnetic beads and/or with reagents for biochemical assays such as nucleic acid amplification and detection.

The terms “filter module”, “filter module assembly”, “interface device” and are used interchangeably throughout and generally refer to a device for filtering a sample. In some embodiments, the device is portable. In some embodiments the device is one-piece. In some embodiments the device is a multi-component assembly. In some embodiments, the filter module assembly includes a syringe-like system built into a system for handling and/or preparing liquid and/or solid samples. In such an embodiment, the syringe-like system includes an output channel configured to act directly or indirectly with a microfluidic device.

The terms “live hinge” or “living hinge” are used interchangeably throughout and refer to a thin flexible hinge (flexure bearing) made from the same material as the two rigid pieces it connects. It is typically thinned or cut to allow the rigid pieces to bend along the line of the hinge.

An embodiment of the invention relates to a device for assaying a biomolecule from a plant sample including: a microfluidic cartridge for assaying a biomolecule from a plant sample, including: a top layer; and a bottom layer spaced apart from the top layer in a generally parallel orientation with respect to the top layer, the bottom layer defining a plurality of wells therein that protrude from a surface of the bottom layer; and a filter module for filtering the plant sample, including a filter body defining: an upper portion including an inlet structure forming an inlet channel; and a bottom portion configured to accept and secure a filter membrane. In such an embodiment, the filter body is configured to accept a microvolume aliquot of the plant sample, the bottom structure includes an outlet structure forming an outlet channel on an outlet side of the filter membrane, and at least one of the plurality of wells includes an assay reagent solution.

An embodiment of the invention relates to the device above, where at least one of the plurality of wells contains a plurality of magnetic beads, and where the plurality of magnetic beads are configured to bind to the biomolecule.

An embodiment of the invention relates to the device above, where the outlet structure is configured to mechanically connect the bottom structure with the inlet of the top layer of the microfluidic cartridge.

An embodiment of the invention relates to the device above, where the filter module is permanently integrated into the top layer.

An embodiment of the invention relates to the device above, where the filter module further includes a cap structure including a plunger complementary to the inlet channel, such that when in use, the plunger occupies the inlet channel.

An embodiment of the invention relates to the device above, where the cap structure is mechanically connected to the filter module.

An embodiment of the invention relates to the device above, where the cap structure is mechanically connected to the filter module including a live hinge.

An embodiment of the invention relates to the device above, where the outlet structure has a length so as to extend into a well in the bottom layer without reaching a bottom of the well.

An embodiment of the invention relates to the device above, where the inlet structure is configured to accept a microvolume aliquot of the plant sample.

An embodiment of the invention relates to the device above, where the upper portion further includes an overspill channel disposed therein in, the overspill channel distinct from the inlet channel.

An embodiment of the invention relates to the device above, further including a filter membrane disposed in the bottom portion, where the filter membrane includes an average ensemble pore size of up to 20 micrometers in diameter.

An embodiment of the invention relates to the device above, where where the inlet structure is configured to mechanically connect to a sample loading device.

An embodiment of the invention relates to the device above, where the filter body is a multi-component assembly including: a filter module for filtering the plant sample and configured to mechanically connect to the microfluidic cartridge, the filter module including: an upper portion including an inlet structure forming an inlet channel; a middle layer configured to accept and secure a filter membrane; and a bottom portion configured to accept the middle layer. In such an embodiment, the upper portion and the bottom portion are configured to couple with one another to form a assembly such that the middle layer is disposed within the fluid-tight assembly during use, the fluid-tight assembly is configured to accept a microvolume aliquot of the plant sample, the bottom portion includes an outlet structure forming an outlet channel on an outlet side of the middle layer, and the outlet structure is configured to mechanically connect the bottom portion with the inlet of the top layer of the microfluidic cartridge.

An embodiment of the invention relates to the device above, further including a filter membrane disposed in the middle layer, where the filter membrane includes an average ensemble pore size of up to 20 micrometers in diameter.

An embodiment of the invention relates to the device above, where at least one of the plurality of wells is a sample well configured to receive the plant sample therein, and where the inlet is configured to provide access to the sample well.

An embodiment of the invention relates to the device above, further including a second filter membrane disposed in the middle layer, such that the second filter membrane is in an anterior orientation during use with respect to the filter membrane.

An embodiment of the invention relates to the device above, where the second filter membrane includes an average ensemble pore size of up to 20 micrometers in diameter.

An embodiment of the invention relates to the device above, where the top layer further forms a pressure relief opening. In some embodiments, the pressure relief opening is adjacent to the inlet.

An embodiment of the invention relates to the device above, where the inlet structure is configured to mechanically connect to a sample loading device.

An embodiment of the invention relates to the device above, where at least one of the plurality of wells is a sample well configured to receive the plant sample therein, the sample well further includes a bead retaining structure configured to descend below a base portion of the sample loading well.

An embodiment of the invention relates to the device above, where at least one of the plurality of wells is an assay well, the assay well configured to operably engage with to a thermocycling element of an assay device. In some embodiments, the assay well is configured to engage with a thermocycling elements of a device for polymerase chain reactions assays.

An embodiment of the invention relates to the device above, where the outlet structure has a length of between 1.1 mm to 6.0 mm.

An embodiment of the invention relates to the device above, where the outlet channel has a diameter of between 0.8 mm to 3.4 mm.

An embodiment of the invention relates to the device above, where the bottom portion has an inner diameter of between 10.0 mm to 25.0 mm.

An embodiment of the invention relates to the device above, where the bottom portion has an outer diameter of between 11.0 mm to 26.0 mm.

An embodiment of the invention relates to the device above, where an inner diameter of the bottom portion and an inner diameter of the outlet channel have a ratio of between 31.25:1 and 1:1.

An embodiment of the invention relates to the device above, where the filter membrane includes a material selected from the group consisting of nylon, Polytetrafluoroethylene (PTFE), cellulose acetate (CA).

An embodiment of the invention relates to the device above, where the device further includes an adapter configured to mechanically connect the filter module to the microfluidic cartridge.

An embodiment of the invention relates to the device above, where the filter membrane has a diameter of between 10.0 mm and 25.0 mm.

An embodiment of the invention relates to the device above, where the biomolecule is a nucleic acid sequence.

An embodiment of the invention relates to the device above, where the filter module is portable.

An embodiment of the invention relates to a filter module for filtering a plant sample, including a fluid-tight filter body defining: an upper portion including an inlet structure forming an inlet channel; and a bottom portion configured to accept and secure a filter membrane. In such an embodiment, the fluid-tight filter body is configured to accept a microvolume aliquot of the plant sample, the bottom portion includes an outlet structure forming an outlet channel on an outlet side of the filter membrane, and the outlet structure is configured to mechanically connect the bottom portion with a microfluidic cartridge.

An embodiment of the invention relates to the filter module above, further including a filter membrane disposed in the bottom portion, where the filter membrane includes an average ensemble pore size of up to 2 micrometers in diameter.

An embodiment of the invention relates to the filter module above, where the inlet structure is configured to mechanically connect to a sample loading device.

An embodiment of the invention relates to the filter module above, where the outlet structure has a length so as to extend into a well in the microfluidic cartridge without reaching a bottom of the well.

An embodiment of the invention relates to the filter module above, where the fluid-tight filter body is a multi-component assembly including: an upper portion including an inlet structure forming an inlet channel; a middle layer configured to accept and secure a filter membrane; and a bottom portion configured to accept the middle layer. In such an embodiment, the upper portion and the bottom portion are configured to couple with one another to form a fluid-tight assembly such that the middle layer is disposed within the fluid-tight assembly during use, the fluid-tight assembly is configured to accept a microvolume aliquot of the plant sample, the bottom portion includes an outlet structure forming an outlet channel on an outlet side of the middle layer, and the outlet structure is configured to mechanically connect the bottom portion with a microfluidic cartridge.

An embodiment of the invention relates to the filter module above, further including a filter membrane disposed in the middle layer, where the filter membrane includes an average ensemble pore size of up to 2 micrometers in diameter.

An embodiment of the invention relates to the filter module above, further including a second filter membrane disposed in the middle layer, such that the second filter membrane is in an anterior orientation during use with respect to the filter membrane.

An embodiment of the invention relates to the filter module above, where the second filter membrane includes an average ensemble pore size of up to 20 micrometers in diameter.

An embodiment of the invention relates to the filter module above, where the filter membrane has a diameter of between 10.0 mm and 25.0 mm.

An embodiment of the invention relates to the filter module above, where the outlet structure has a length of between 1.1 mm to 6.0 mm.

An embodiment of the invention relates to the filter module above, where the outlet channel has a diameter of between 0.8 mm to 3.4 mm.

An embodiment of the invention relates to the filter module above, where the bottom portion has an inner diameter of between 10.0 mm to 25.0 mm.

An embodiment of the invention relates to the filter module above, where the bottom portion has an outer diameter of between 11.0 mm to 26.0 mm.

An embodiment of the invention relates to the filter module above, where an inner diameter of the bottom portion and an inner diameter of the outlet channel have a ratio of between 31.25:1 and 1:1.

An embodiment of the invention relates to the filter module above, where the filter membrane includes a material selected from the group consisting of nylon, Polytetrafluoroethylene (PTFE), cellulose acetate (CA).

An embodiment of the invention relates to the filter module above, where the filter module is portable.

An embodiment of the invention relates a method of detecting a biomolecule in a plant sample, including: preparing a lysate including the plant sample by contacting the plant sample with a lysis buffer; filtering a microvolume aliquot of the lysate using a filter module; loading the filtered plant sample into a sample well of a microfluidic cartridge; amplifying the biomolecule; and detecting the biomolecule. In such an embodiment, preparing the lysate and the filtering the microvolume aliquot of the lysate are done at an ambient temperature.

An embodiment of the invention relates to the method above, where the filter module includes: an upper portion including an inlet structure forming an inlet channel; and a bottom portion configured to accept and secure a filter membrane. In such an embodiment, the filter assembly is configured to accept a microvolume aliquot of the plant sample in the inlet channel, the bottom portion includes an outlet structure forming an outlet channel on an outlet side of the filter membrane, and the outlet structure has a length so as to extend into a well in the bottom layer without reaching a bottom of the well.

An embodiment of the invention relates to the method above, where the filter module includes: an upper portion including an inlet structure forming an inlet channel; a middle layer configured to accept and secure a filter membrane; and a bottom portion configured to accept the middle layer. In such an embodiment, the upper portion and the bottom portion are configured to couple with one another to form a fluid-tight assembly such that the middle layer is disposed within the fluid-tight assembly during use, the fluid-tight assembly is configured to accept a microvolume aliquot of the plant sample, the bottom portion includes an outlet structure forming an outlet channel on an outlet side of the middle layer, and the outlet structure is configured to mechanically connect the bottom portion with an inlet formed by a top layer of the microfluidic cartridge.

An embodiment of the invention relates to the method above, where the filter module further includes a filter membrane disposed in the middle layer, where the filter membrane includes an average ensemble pore size of up to 20 micrometers in diameter.

An embodiment of the invention relates to the method above, where the lysis buffer has a pH of between 3.6 and 6.5.

An embodiment of the invention relates to the method above, where preparing a lysate and the filtering a microvolume aliquot of the lysate occur in under 1 to 10 minutes.

An embodiment of the invention relates to the method above, where the filter module is portable.

An embodiment of the invention relates to a method of detecting a biomolecule in a plant sample, including the steps of: preparing a lysate including the plant sample by contacting the plant sample with a lysis buffer; filtering a microvolume aliquot of the lysate using a filter module; loading the filtered plant sample into a sample well of a microfluidic cartridge; amplifying the biomolecule; and detecting the biomolecule. In such an embodiment, the step of preparing the lysate and the filtering the microvolume aliquot of the lysate are done at an ambient temperature.

An embodiment of the invention relates to the method above, where the filter module includes: an upper portion including an inlet structure forming an inlet channel; a middle layer configured to accept and secure a filter membrane; and a bottom portion configured to accept the middle layer. The upper portion and the bottom portion are configured to couple with one another to form a fluid-tight assembly such that the middle layer is disposed within the fluid-tight assembly during use. The fluid-tight assembly is configured to accept a microvolume aliquot of the plant sample. The bottom portion includes an outlet structure forming an outlet channel on an outlet side of the middle layer. The outlet structure is configured to mechanically connect the bottom portion with an inlet formed by a top layer of the microfluidic cartridge.

An embodiment of the invention relates to the method above, where the filter module further includes a filter membrane disposed in the middle layer. The filter membrane includes an average ensemble pore size of up to 2 micrometers in diameter.

An embodiment of the invention relates to the method above, where the lysis buffer has a pH of between 3.6 and 6.5.

An embodiment of the invention relates to the method above, where the preparing a lysate and the filtering a microvolume aliquot of the lysate occur in under 1 to 10 minutes.

An embodiment of the invention relates to the method above, where the filter module is portable.

An embodiment of the invention relates to a filter module for filtering a plant sample, having a fluid-tight filter body defining: an upper portion including an inlet structure forming an inlet channel; and a bottom portion configured to accept and secure a filter membrane. The fluid-tight filter body is configured to accept a microvolume aliquot of the plant sample. The bottom portion includes an outlet structure forming an outlet channel on an outlet side of the filter membrane. The outlet structure is configured to mechanically connect the bottom portion with a microfluidic cartridge.

An embodiment of the invention relates to the filter module above, further including a filter membrane disposed in the bottom portion, where the filter membrane includes an average ensemble pore size of up to 2 micrometers in diameter.

An embodiment of the invention relates to the filter module above, where the inlet structure is configured to mechanically connect to a syringe.

An embodiment of the invention relates to the filter module above, where the outlet structure has a length so as to extend into a well in the microfluidic cartridge without reaching a bottom of the well.

An embodiment of the invention relates to the filter module above, where the fluid-tight filter body is a multi-component assembly having: an upper portion including an inlet structure forming an inlet channel; a middle layer configured to accept and secure a filter membrane; and a bottom portion configured to accept the middle layer. The upper portion and the bottom portion are configured to couple with one another to form a fluid-tight assembly such that the middle layer is disposed within the fluid-tight assembly during use. The fluid-tight assembly is configured to accept a microvolume aliquot of the plant sample. The bottom portion includes an outlet structure forming an outlet channel on an outlet side of the middle layer. The outlet structure is configured to mechanically connect the bottom portion with a microfluidic cartridge.

An embodiment of the invention relates to the filter module above, further having a filter membrane disposed in the middle layer, where the filter membrane includes an average ensemble pore size of up to 2 micrometers in diameter.

An embodiment of the invention relates to the filter module above, further having a second filter membrane disposed in the middle layer, such that the second filter membrane is in an anterior orientation during use with respect to the filter membrane.

An embodiment of the invention relates to the filter module above, where the second filter membrane includes an average ensemble pore size of up to 20 micrometers in diameter.

An embodiment of the invention relates to the filter module above, where the filter membrane has a diameter of between 10.0 mm and 25.0 mm.

An embodiment of the invention relates to the filter module above, where the outlet structure has a length of between 1.1 mm to 6.0 mm.

An embodiment of the invention relates to the filter module above, where the outlet channel has a diameter of between 0.8 mm to 3.4 mm.

An embodiment of the invention relates to the filter module above, where the bottom portion has an inner diameter of between 10.0 mm to 25.0 mm.

An embodiment of the invention relates to the filter module above, where the bottom portion has an outer diameter of between 11.0 mm to 26.0 mm.

An embodiment of the invention relates to the filter module above, where an inner diameter of the bottom portion and an inner diameter of the outlet channel have a ratio of between 31.25:1 and 1:1.

An embodiment of the invention relates to the filter module above, where the filter membrane includes a material selected from the group consisting of nylon, Polytetrafluoroethylene (PTFE), cellulose acetate (CA).

An embodiment of the invention relates to the filter module above, where the filter module is portable.

An embodiment of the invention relates to a device for assaying a nucleic acid sequence from a plant sample having: a microfluidic cartridge for assaying a nucleic acid sequence from a plant sample, having: a top layer forming an inlet; and a bottom layer spaced apart from the top layer in a generally parallel orientation with respect to the top layer, the bottom layer defining a plurality of wells therein that protrude from a surface of the bottom layer; and a filter module for filtering the plant sample, including a fluid-tight filter body defining: an upper portion including an inlet structure forming an inlet channel; and a bottom portion configured to accept and secure a filter membrane. The fluid-tight filter body is configured to accept a microvolume aliquot of the plant sample. The bottom portion includes an outlet structure forming an outlet channel on an outlet side of the filter membrane. The outlet structure is configured to mechanically connect the bottom portion with the inlet of the top layer of the microfluidic cartridge.

An embodiment of the invention relates to the device above, further having a filter membrane disposed in the bottom portion, where the filter membrane includes an average ensemble pore size of up to 2 micrometers in diameter.

An embodiment of the invention relates to the device above, where the inlet structure is configured to mechanically connect to a syringe.

An embodiment of the invention relates to the device above, where the outlet structure has a length so as to extend into a well in the microfluidic cartridge without reaching a bottom of the well.

An embodiment of the invention relates to the device above, where the fluid-tight filter body is a multi-component assembly includes: a filter module for filtering the plant sample and configured to mechanically connect to the microfluidic cartridge, the filter module having: an upper portion including an inlet structure forming an inlet channel; a middle layer configured to accept and secure a filter membrane; and a bottom portion configured to accept the middle layer. The upper portion and the bottom portion are configured to couple with one another to form a fluid-tight assembly such that the middle layer is disposed within the fluid-tight assembly during use. The fluid-tight assembly is configured to accept a microvolume aliquot of the plant sample. The bottom portion includes an outlet structure forming an outlet channel on an outlet side of the middle layer. The outlet structure is configured to mechanically connect the bottom portion with the inlet of the top layer of the microfluidic cartridge.

An embodiment of the invention relates to the device above, further having a filter membrane disposed in the middle layer, where the filter membrane includes an average ensemble pore size of up to 2 micrometers in diameter.

An embodiment of the invention relates to the device above, where at least one of the plurality of wells is a sample well configured to receive the plant sample therein, and where the inlet is configured to provide access to the sample well.

An embodiment of the invention relates to the device above, further having a second filter membrane disposed in the middle layer, such that the second filter membrane is in an anterior orientation during use with respect to the filter membrane.

An embodiment of the invention relates to the device above, where the second filter membrane includes an average ensemble pore size of up to 20 micrometers in diameter.

An embodiment of the invention relates to the device above, where the top layer further forms a pressure relief opening adjacent to the inlet.

An embodiment of the invention relates to the device above, where the inlet structure is configured to mechanically connect to a syringe.

An embodiment of the invention relates to the device above, where the filter membrane has a diameter of between 10.0 mm and 25.0 mm.

An embodiment of the invention relates to the device above, where the outlet structure has a length of between 1.1 mm to 6.0 mm.

An embodiment of the invention relates to the device above, where outlet channel has a diameter of between 0.8 mm to 3.4 mm.

An embodiment of the invention relates to the device above, where the bottom portion has an inner diameter of between 10.0 mm to 25.0 mm.

An embodiment of the invention relates to the device above, where the bottom portion has an outer diameter of between 11.0 mm to 26.0 mm.

An embodiment of the invention relates to the device above, where an inner diameter of the bottom portion and an inner diameter of the outlet channel have a ratio of between 31.25:1 and 1:1.

An embodiment of the invention relates to the device above, where the filter membrane includes a material selected from the group consisting of nylon, Polytetrafluoroethylene (PTFE), cellulose acetate (CA).

An embodiment of the invention relates to the device above, where the device further includes an adapter configured to mechanically connect the filter module to the microfluidic cartridge.

An embodiment of the invention relates to the device above, where at least one of the plurality of wells contains a plurality of magnetic beads, and where the plurality of magnetic beads are configured to bind to the nucleic acid sequence.

An embodiment of the invention relates to the device above, where the filter module is portable.

An embodiment of the invention relates to a method of detecting a biomolecule in a plant sample, including the steps of: preparing a lysate including the plant sample by contacting the plant sample with a lysis buffer; filtering a microvolume aliquot of the lysate using a filter module; loading the filtered plant sample into a sample well of a microfluidic cartridge; amplifying the biomolecule; and detecting the biomolecule. In such an embodiment, the steps of preparing the lysate and the filtering the microvolume aliquot of the lysate are done at an ambient temperature.

An embodiment of the invention relates to the method of detecting a biomolecule in a plant sample above, where the filter module includes: an upper portion including an inlet structure forming an inlet channel; a middle layer configured to accept and secure a filter membrane; and a bottom portion configured to accept the middle layer. In such an embodiment, the upper portion and the bottom portion are configured to couple with one another to form a fluid-tight assembly such that the middle layer is disposed within the assembly during use, the assembly is configured to accept a microvolume aliquot of the plant sample, the bottom portion includes an outlet structure forming an outlet channel on an outlet side of the middle layer, and the outlet structure is configured to mechanically connect the bottom portion with an inlet formed by a top layer of the microfluidic cartridge.

An embodiment of the invention relates to the method of detecting a biomolecule in a plant sample above, where the filter module further has a filter membrane disposed in the middle layer, where the filter membrane has an average ensemble pore size of up to 2 micrometers in diameter.

An embodiment of the invention relates to the method of detecting a biomolecule in a plant sample above, where the lysis buffer has a pH of between 3.6 and 6.5.

An embodiment of the invention relates to the method of detecting a biomolecule in a plant sample above, where the steps of preparing a lysate and filtering a microvolume aliquot of the lysate occur in under 1 to 10 minutes.

An embodiment of the invention relates to a filter module for filtering a plant sample, including: an upper portion including an inlet structure forming an inlet channel; a middle layer configured to accept and secure a filter membrane; and a bottom portion configured to accept the middle layer. In such an embodiment, the upper portion and the bottom portion are configured to couple with one another to form a fluid-tight assembly such that the middle layer is disposed within the assembly during use, the assembly is configured to accept a microvolume aliquot of the plant sample, the bottom portion has an outlet structure forming an outlet channel on an outlet side of the middle layer, and the outlet structure is configured to mechanically connect the bottom portion with a microfluidic cartridge.

An embodiment of the invention relates to the filter module above, further having a filter membrane disposed in the middle layer, where the filter membrane includes an average ensemble pore size of up to 2 micrometers in diameter.

An embodiment of the invention relates to the filter module above, where the inlet structure is configured to mechanically connect to a syringe.

An embodiment of the invention relates to the filter module above, where the outlet structure has a length so as to extend into a well in the microfluidic cartridge without reaching a bottom of the well.

An embodiment of the invention relates to the filter module above, further having a second filter membrane disposed in the middle layer, such that the second filter membrane is in an anterior orientation during use with respect to the filter membrane.

An embodiment of the invention relates to the filter module above, where the second filter membrane includes an average ensemble pore size of up to 20 micrometers in diameter.

An embodiment of the invention relates to the filter module above, where the filter layer has a diameter of between 10.0 mm and 25.0 mm.

An embodiment of the invention relates to the filter module above, where the outlet structure has a length of between 1.1 mm to 6.0 mm.

An embodiment of the invention relates to the filter module above, where the outlet channel has a diameter of between 0.8 mm to 3.4 mm.

An embodiment of the invention relates to the filter module above, where the bottom portion has an inner diameter of between 10.0 mm to 25.0 mm.

An embodiment of the invention relates to the filter module above, where the bottom portion has an outer diameter of between 11.0 mm to 26.0 mm.

An embodiment of the invention relates to the filter module above, where an inner diameter of the bottom portion and an inner diameter of the outlet channel have a ratio of between 31.25:1 and 1:1.

An embodiment of the invention relates to the filter module above, where the filter membrane includes a material selected from the group consisting of nylon, Polytetrafluoroethylene (PTFE), cellulose acetate (CA).

An embodiment of the invention relates to the filter module above, where the filter remains operable in a pH of about 3.6 to a pH of about 6.5.

An embodiment of the invention relates to a device for assaying a nucleic acid sequence from a plant sample having: a microfluidic cartridge for assaying a nucleic acid sequence from a plant sample, having: a top layer forming an inlet; and a bottom layer spaced apart from the top layer in a generally parallel orientation with respect to the top layer, the bottom layer defining a plurality of wells therein that protrude from a surface of the bottom layer; and a filter module for filtering the plant sample and configured to mechanically connect to the microfluidic cartridge, the filter module having: an upper portion including an inlet structure forming an inlet channel; a middle layer configured to accept and secure a filter membrane; and a bottom portion configured to accept the middle layer. In such an embodiment, the upper portion and the bottom portion are configured to couple with one another to form a fluid-tight assembly such that the middle layer is disposed within the assembly during use, the assembly is configured to accept a microvolume aliquot of the plant sample, the bottom portion includes an outlet structure forming an outlet channel on an outlet side of the middle layer, and the outlet structure is configured to mechanically connect the bottom portion with the inlet of the top layer of the microfluidic cartridge.

An embodiment of the invention relates to the device for assaying a nucleic acid sequence from a plant sample above, further having a filter membrane disposed in the middle layer, where the filter membrane has an average ensemble pore size of up to 2 micrometers in diameter.

An embodiment of the invention relates to the device for assaying a nucleic acid sequence from a plant sample above, where at least one of the plurality of wells is a sample well configured to receive the plant sample therein, and where the inlet is configured to provide access to the sample well.

An embodiment of the invention relates to the device for assaying a nucleic acid sequence from a plant sample above, where the top layer further forms a pressure relief opening adjacent to the inlet. In an embodiment, the pressure relief opening is a vent.

An embodiment of the invention relates to the device for assaying a nucleic acid sequence from a plant sample above, where the inlet structure is configured to mechanically connect to a syringe.

An embodiment of the invention relates to the device for assaying a nucleic acid sequence from a plant sample above, where the inlet structure is configured to mechanically connect to a syringe.

An embodiment of the invention relates to the device for assaying a nucleic acid sequence from a plant sample above, where the outlet structure has a length so as to extend into a well in the microfluidic cartridge without reaching a bottom of the well.

An embodiment of the invention relates to the device for assaying a nucleic acid sequence from a plant sample above, further having a second filter membrane disposed in the middle layer, such that the second filter membrane is in an anterior orientation during use with respect to the filter membrane.

An embodiment of the invention relates to the device for assaying a nucleic acid sequence from a plant sample above, where the second filter membrane includes an average ensemble pore size of up to 20 micrometers in diameter.

An embodiment of the invention relates to the device for assaying a nucleic acid sequence from a plant sample above, where the filter layer has a diameter of between 10.0 mm and 25.0 mm.

An embodiment of the invention relates to the device for assaying a nucleic acid sequence from a plant sample above, where the outlet structure has a length of between 1.1 mm to 6.0 mm.

An embodiment of the invention relates to the device for assaying a nucleic acid sequence from a plant sample above, where the outlet channel has a diameter of between 0.8 mm to 3.4 mm.

An embodiment of the invention relates to the device for assaying a nucleic acid sequence from a plant sample above, where the bottom portion has an inner diameter of between 10.0 mm to 25.0 mm.

An embodiment of the invention relates to the device for assaying a nucleic acid sequence from a plant sample above, where the bottom portion has an outer diameter of between 111.0 mm to 26.0 mm.

An embodiment of the invention relates to the device for assaying a nucleic acid sequence from a plant sample above, where an inner diameter of the bottom portion and an inner diameter of the outlet channel have a ratio of between 31.25:1 and 1:1.

An embodiment of the invention relates to the device for assaying a nucleic acid sequence from a plant sample above, where the filter membrane includes a material selected from the group consisting of nylon, Polytetrafluoroethylene (PTFE), cellulose acetate (CA).

An embodiment of the invention relates to the device for assaying a nucleic acid sequence from a plant sample above, where the filter remains operable in a pH of about 3.6 to a pH of about 6.5.

Some embodiments of the invention relate to methods for the genetic testing of plant materials outside conventional laboratory testing sites, for applications including but not limited to allelic discrimination and genetic biomarker quantification. Some features of such embodiments include but are not limited to methods of utilizing plant material for nucleic acid analysis using a three-step process including the steps of: 1) plant cell lysis and expression of nucleic acids in solution by the use of chemical reagents, from starting plant material including but not limited to ground seed and punched seed; 2) the use of one or more filtration devices to separate a solution containing nucleic acids from particulates; and 3) the use of the solution from the second step for the analysis of nucleic acids in a microfluidic cartridge.

Some embodiments of the invention relate to processes to perform allelic discrimination and quantitative nucleic acid testing in absence of standard laboratory equipment.

Some embodiments of the current invention are directed to methods and devices for assaying a nucleic acid sequence or other biomolecule using a plurality of magnetic beads and a plurality of magnets positioned around the device. Briefly, in such embodiments, magnetic beads are deposited into a sample well; these magnetic beads are configured to bind to the nucleic acid. Once bound to the magnetic beads, the nucleic acid is then transported from the sample well to one or more downstream wells for assaying by actuation of the magnets. More specifically, one or more magnetic particles are manipulated in two dimensions. The first dimension is defined by the extent of transverse motion of magnetic particles between the innermost part of the extruded feature and the planar hydrophobic substrate. The second dimension is defined by the extent of longitudinal motion of magnetic particles along the planar hydrophobic substrate. Particle extraction, translocation and re-suspension facilitated by magnetic actuation in a combination of the two dimensions, where a two-axis mechanical manipulator is an embodiment. Additional specifics of such methods are described in U.S. Pat. No. 9,463,461 and Published International Patent Application PCT/US2019/029937, which are hereby incorporated by reference.

FIGS. 2A and 2B are schematics showing a device 1801 for assaying a biomolecule from a plant sample according to an embodiment of the invention. In FIGS. 2A and 2B, the device 1801 includes a top layer 1803, and a bottom layer 1805 spaced apart from the top layer 1803 in a generally parallel orientation with respect to the top layer 1803. The bottom layer has one or more wells 1807 that protrude from the surface of the bottom layer 1805. The device also contains a permanently integrated filter module 1809 for filtering the plant sample. The filter module has an upper portion 1810 having an inlet structure forming an inlet channel 1811, and a bottom portion 1812 configured to accept and secure a filter membrane 1813. The filter module 1809 is configured to accept a microvolume aliquot of the plant sample. The filter module 1809 also includes a cap 1815 mechanically connected to the filter module 1809 by a live hinge 1817. The cap 1815 also has a plunger 1819 complementary to the inlet channel and configured to fill the inlet channel when the cap structure is in use. By closing the cap 1815, a user actuates the plunger 1819 and allows for the plant sample to pass through the filter membrane 1813 and into a well 1807. The top layer 1803 also includes an open port 1821 for the loading of silicone oil into the device prior to use. The filter module 1809 also includes an overspill channel 1820 for allowing displaced fluids from sample introduction to be captured. At least the first of the plurality of wells contains reagents for a biological assay. In some embodiments, this well also contains magnetic beads configured to bind to the biomolecule.

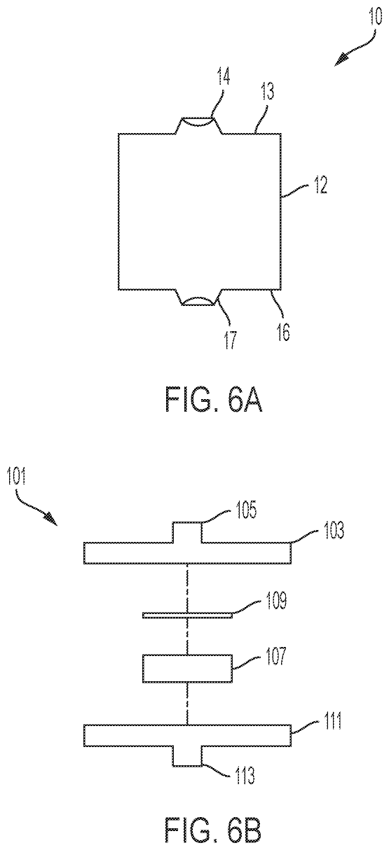

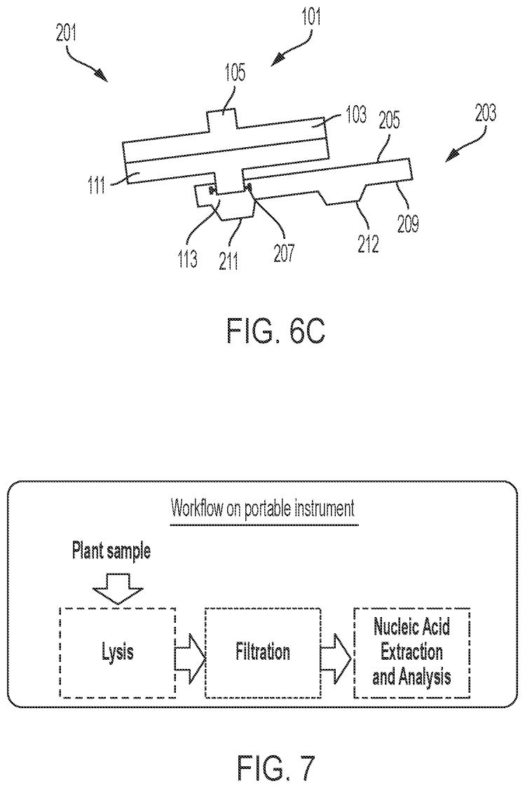

FIG. 6A is a schematic depicting a filter module 10 for filtering a plant sample according to an embodiment of the invention. The filter module 10 has a fluid-tight filter body 12 defining: an upper portion 13 including an inlet structure 14 forming an inlet channel; and a bottom portion 16 configured to accept and secure a filter membrane (not shown). The fluid-tight filter body 12 is configured to accept a microvolume aliquot of the plant sample. The bottom portion 16 includes an outlet structure 17 forming an outlet channel on an outlet side of the filter membrane. The outlet structure 17 is configured to mechanically connect the bottom portion 16 with a microfluidic cartridge (not shown).

FIG. 6B is a schematic depicting a filter module 101 according to an embodiment of the invention. The filter module 101 for filtering a plant sample includes an upper portion 103 including an inlet structure 105 forming an inlet channel. It also includes a middle layer 107 configured to accept and secure a filter membrane 109. It also includes a bottom portion 111 configured to accept the middle layer 107. In such an embodiment, the upper portion 103 and the bottom portion 111 are configured to couple with one another to form a fluid-tight assembly such that the middle layer is disposed within the assembly during use. The assembly is configured to accept a microvolume aliquot of the plant sample. The bottom portion 111 has an outlet structure 113 forming an outlet channel on an outlet side of the middle layer. The outlet structure is configured to mechanically connect the bottom portion with a microfluidic cartridge (not shown).

FIG. 6C is a schematic depicting an embodiment of the invention. FIG. 6C shows a device 201 for assaying a nucleic acid sequence from a plant sample having a microfluidic cartridge 203 for assaying a nucleic acid sequence from a plant sample. The microfluidic cartridge has a top layer 205 forming an inlet 207. The microfluidic cartridge also has a bottom layer 209 spaced apart from the top layer 205 in a generally parallel orientation with respect to the top layer 205. The bottom layer 209 defines a plurality of wells 211, 212 therein that protrude from a surface of the bottom layer 209. The device 201 aslo includes a filter module 101 for filtering the plant sample and configured to mechanically connect to the microfluidic cartridge 203. The filter module has an upper portion 103 including an inlet structure 105 forming an inlet channel. The filter module also has a middle layer (shown in FIG. 6B) configured to accept and secure a filter membrane (shown in FIG. 6B). The filter module also has a bottom portion 111 configured to accept the middle layer. In such an embodiment, the upper portion 103 and the bottom portion 111 are configured to couple with one another to form a fluid-tight assembly such that the middle layer is disposed within the assembly during use. The assembly is configured to accept a microvolume aliquot of the plant sample. The bottom portion 111 includes an outlet structure 113 forming an outlet channel on an outlet side of the middle layer, and the outlet structure is configured to mechanically connect the bottom portion 111 with the inlet 207 of the top layer 205 of the microfluidic cartridge 203.

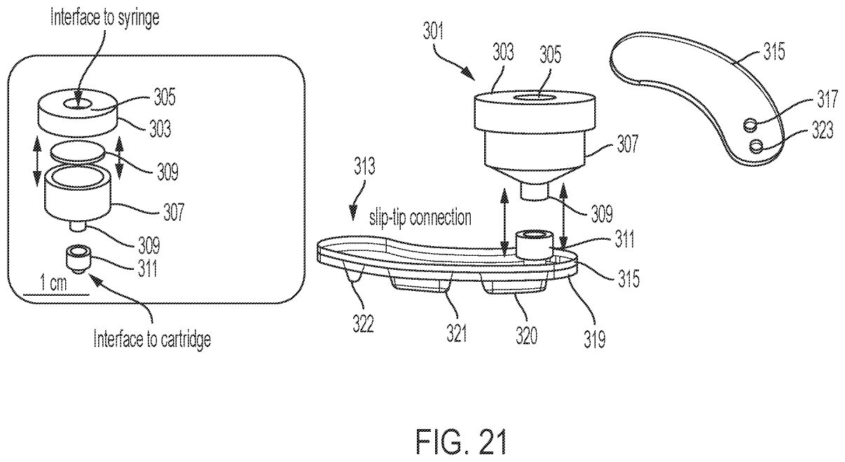

FIG. 21 is a schematic depicting an embodiment of the invention. FIG. 21 shows a filter module 301 for filtering a plant sample includes an upper portion 303 including an inlet structure 305 forming an inlet channel. It also includes a bottom portion 307 configured to accept and secure a filter membrane 309. In such an embodiment, the upper portion 303 and the bottom portion 307 are configured to couple with one another to form a fluid-tight assembly such that the filter membrane is disposed within the assembly during use. The assembly is configured to accept a microvolume aliquot of the plant sample. The bottom portion 307 has an outlet structure 309 forming an outlet channel on an outlet side of the filter membrane. The outlet structure is configured to mechanically connect the bottom portion with an adapter 311, which is in turn configured to connect to a microfluidic cartridge 313. The microfluidic cartridge has a top layer 315 forming an inlet 317. The microfluidic cartridge also has a bottom layer 319 spaced apart from the top layer 315 in a generally parallel orientation with respect to the top layer 315. The bottom layer 319 defines a plurality of wells 320, 321, 322 therein that protrude from a surface of the bottom layer 319. The adapter 311 is configured to connect the bottom portion 307 of the fluid assembly with the inlet 317 of the microfluidic cartridge 313. The top layer 315 of the microfluidic cartridge 313 also defines another opening 323, which acts as a vent where excess air is pushed out during loading of the microfluidic cartridge 313.

### EXAMPLES

The following describes some concepts of the current invention with reference to particular examples. The general concepts of the current invention are not limited to the examples described.

### Example 1

There is a need for a device that can connect a reservoir containing plant lysate with an assay platform. In such an embodiment, the device should also provide the removal function of the precipitants in plant lysate when the lysate is transferred from the reservoir into the assay platform. The embodiment depicted in FIGS. 2A-FIG. 5 these issues.

The device of FIGS. 2A-FIG. 5 utilizes an integrated filtration system in a cartridge, which removes particulates from a sample solution to enable real-time detection of DNA markers via probe-based real-time nucleic acid amplification testing assay. The device shown can be used for integrating plant lysate input to the downstream assay platform.

In such a device, an embodiment of the filtration system includes the top cap of the assay cartridge. The loading well of the cartridge contains an integrated sample filtration matrix over a narrow nozzle tip going into the input well of a cartridge. A sealing cap includes a plunger to fill space in the loading well when closed to force the sample through the filtration matrix. On the opposite end of the cartridge cap, there is an open port for the loading of silicone oil into the cartridge prior to use.

In some assays, magnetic beads are pre-loaded into the cartridge, such as on the filter matrix, in the nozzle after the filter matrix, or in the first well of the cartridge underneath the nozzle. In addition, beads could be mixed into the plant lysate as well. In embodiments where the beads are pre-loaded into the cartridge, the loading well may include an additional bead retaining structure (see for example FIG. 24, structure 2401) that descends below the base of the loading well and secures the beads in that position during storage and shipment. The outlet channel 1823 may be optimally positioned to be above or proximal to the bead retaining structure.

To use the device, a sample of plant lysate is loaded into the input well on top of the filter assembly. The sealing cap is then inserted into the input well, using the plunger to force the sample through the filter. The sample travels through the filter and is deposited by the nozzle into the bottom of the first well of the cartridge.

In an embodiment according to the invention, the filtration system materials are modified to avoid the biomolecule of interest from sticking or accumulating on to surfaces as the sample passes through. The pore size and material of filter membrane depend upon the particular use, and the preferred material of filter membrane should be able to sustain high acidic solution and low static charge to prevent the loss of nucleic acid. In this particular use of genotyping using nucleic acid from plant lysate, pore size of filter should not be smaller than 2.0 μm to ensure the yield of nucleic acid after filtration. Furthermore, a second layer of filter membrane with pore size larger than the first/finer filter membrane can be added to remove the larger cellular debris before lysate reaches the finer filter membrane. The material of the second filter layer can be nylon as an example.

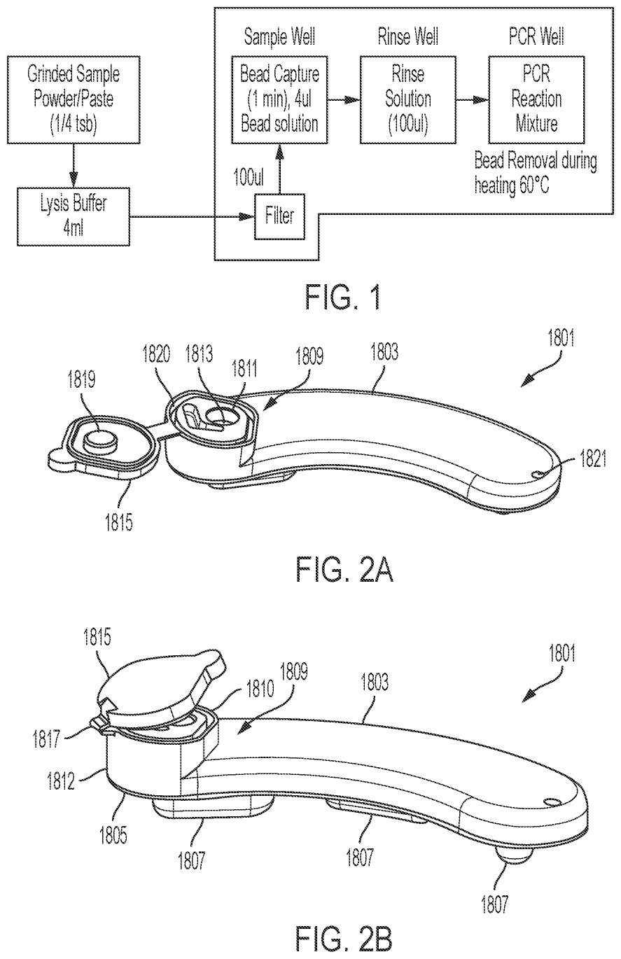

FIG. 1 is a schematic showing a general approach for using a device having an integrated filter module. In FIG. 1, the sample is contacted with a lysis buffer before being passed through a filter membrane in the filter module. The sample is then pushed into the sample well. The sample is then passed through a rinsing well, and ultimately passed into a well for polymerase chain reaction (PCR).

FIGS. 2A and 2B are schematics showing a device 1801 for assaying a biomolecule from a plant sample. In FIGS. 2A and 2B, the device 1801 includes a top layer 1803, and a bottom layer 1805 spaced apart from the top layer 1803 in a generally parallel orientation with respect to the top layer 1803. The bottom layer has one or more wells 1807 that protrude from the surface of the bottom layer 1805. The device also contains a permanently integrated filter module 1809 for filtering the plant sample. The filter module has an upper portion 1810 having an inlet structure forming an inlet channel 1811, and a bottom portion 1812 configured to accept and secure a filter membrane 1813. The filter module 1809 is configured to accept a microvolume aliquot of the plant sample. The filter module 1809 also includes a cap 1815 mechanically connected to the filter module 1809 by a live hinge 1817. The cap 1815 also has a plunger 1819 complementary to the inlet channel and configured to fill the inlet channel when the cap structure is in use. By closing the cap 1815, a user actuates the plunger 1819 and allows for the plant sample to pass through the filter membrane 1813 and into a well 1807. The top layer 1803 also includes an open port 1821 for the loading of silicone oil into the device prior to use. The filter module 1809 also includes an overspill channel 1820 for allowing displaced fluids from sample introduction to be captured. At least the first of the plurality of wells contains reagents for a biological assay. In some embodiments, this well also contains magnetic beads configured to bind to the biomolecule.

FIG. 2A shows a first conformation of the device, where the cap 1815 is removed from the filter module 1809, thus allowing access to the inlet channel 1811. FIG. 2B shows a second conformation of the device where the cap 1815 is in the process of being placed over the filter module 1809.

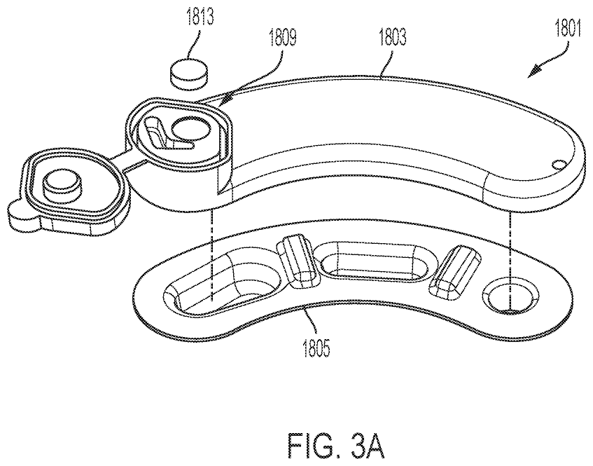

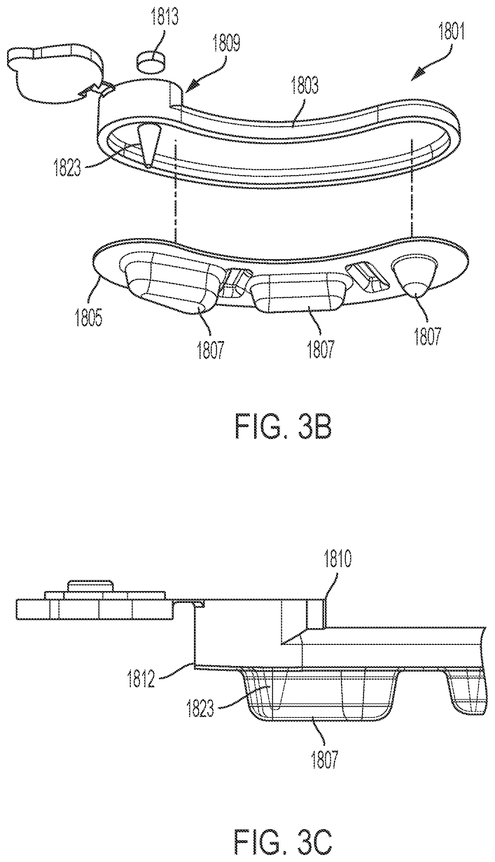

FIGS. 3A and 3B are exploded top and bottom views, respectively, of the device of FIGS. 2A and 2B. As can be seen in FIG. 3B, the device also includes an outlet structure forming an outlet channel 1823 on an outlet side of the filter membrane.

As shown in FIG. 3C, the outlet channel 1823 has a length so as to extend into a well 1807 in the bottom layer 1805 without reaching the bottom of the well 1807.

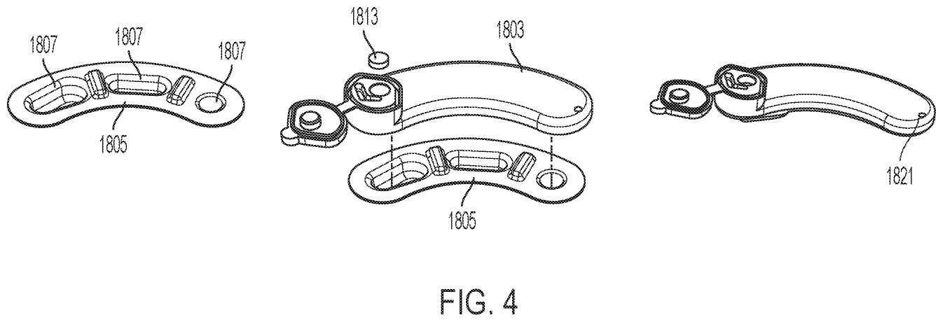

FIG. 4 is a series of images showing how the top layer 1803 and the bottom layer 1805 are combined to form the device 1801. First, the image to the far left shows the bottom layer 1805 sans the top layer 1803. Assay reagents are loaded into the various wells 1807. Next, as shown in the middle panel, the top layer 1803 is placed over the bottom layer 1805, and the tope layer 1803 and bottom layer 1805 are sealed. In some cases, heat is used to form the seal. The seal formed is fluid-tight. Finally, as shown in the far right image, an oil is loaded into the open port 1821 to seal the wells 1807 such that the assay reagents do not spill during shipment and/or use.

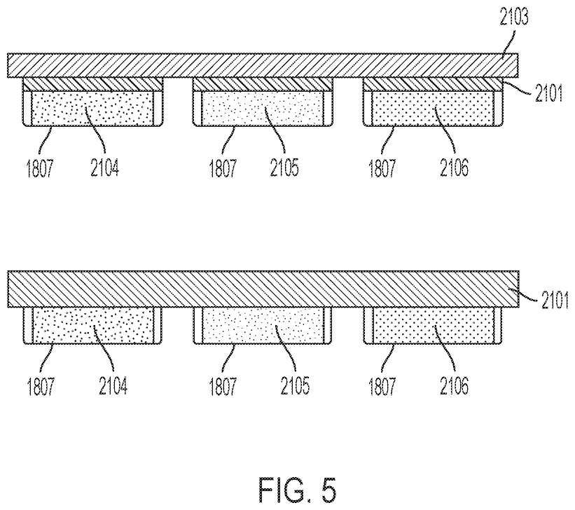

FIG. 5 is a schematic showing the use of wax 2101 and oil 2103 (top image) or wax 2101 (bottom image) to form a seal over various reagents 2104, 2105, 2106 deposited into the wells 1807 of the device of FIGS. 2A and 2B. In some embodiments, magnetic beads are included in one or more of the wells. The magnetic beads are configured to bind to a biomolecule. In such an embodiment, a sample well and an assay well are filled about a ⅓ full with liquid (e.g. one or more reagents, buffers, or rinse fluids) and then covered with wax and/or oil. Examples of suitable waxes and oils to form a seal over the reagents are known to one of ordinary skill in the art. Such suitable waxes and oils are discussed, for example in Sur et al. Immiscible Phase Nucleic Acid Purification Eliminates PCR Inhibitors with a Single Pass of Paramagnetic Particles through a hydrophobic Liquid. Journal of Molecular Diagnostics, Vol. 12, No. 5 (2010), which is hereby incorporated by reference.

### Example 2

As discussed above, the absence of a field-deployable solution to performing genetic analysis of plants leads to logistical challenges for plant trait screening in remote locations around the globe. Recent innovation in assay miniaturization and integration via droplet magnetofluidics create an opportunity to surmount these technical challenges. Magnetofluidic technology replaces bulk fluid transport with magnetic particle manipulation through static discrete microliter droplets, enabling integration of bioassays without the need for complex fluidic cartridges and supporting instrumentation. Magnetic particles are capable of transporting, mixing and separating liquid reagents on small devices ranging from glass-based substrates [Zhang et al., Adv Mater 2014] to thermoplastic cartridges [Shin et al., Sci Rep 2017], facilitating a novel approach to miniaturize and integrate laboratory-bound processes such as nucleic acid extraction on a single device that.

Some embodiments of the invention relate to devices for use in a laboratory-free method for the genetic analysis of plant material at remote testing sites. Briefly, sample is pre-processed into a liquid phase carrying plant nucleic acids by using chemical lysis reagents, followed by removal of plant debris using a filter. In some embodiments, the filtered solution is processed on a disposable cartridge via magnetofluidic sample processing, which enables the necessary purification of nucleic acid targets from crude biosamples to obtain quantitative and consistent assay results. However, one of ordinary skill in the art can readily envision the use of other suitable microfluidic devices.

Methods and Results

Method Overview

Two of the major technical bottlenecks in the implementation of genetic testing include (i) laboratory-dependent sample processing steps for nucleic acid purification and (ii) the need for trained personnel to operate instruments for complex biological assays.

An embodiment of the invention overcomes these issues by implementing (i) a simplified, ambient temperature-compatible protocol for plant cell lysis, consisting of lysis reagents and a filter to separate large particles from the nucleic acid-containing solution; and (ii) automating nucleic acid purification and detection on a portable device by using integrated microfluidic approaches, such as droplet magnetofluidic assay platforms. As a result, it is possible to make the entire assay process portable and reduce assay hands-on time from >1 h to less than 10 minutes (FIG. 7).

As outlined in FIG. 7, a method for genetic analysis of plant specimen according to an embodiment of the invention enables laboratory-free detection of plant biomarkers by using three steps: (i) a lysis process consisting only of lysis reagents at room-temperature, (ii) a filtration process for debris removal, obviating the need for laboratory-bound instruments; and (ii) a method of nucleic acid purification and analysis using a portable droplet magnetofluidic assay device, which reduces hands-on sample processing time and enables analysis in a portable format.

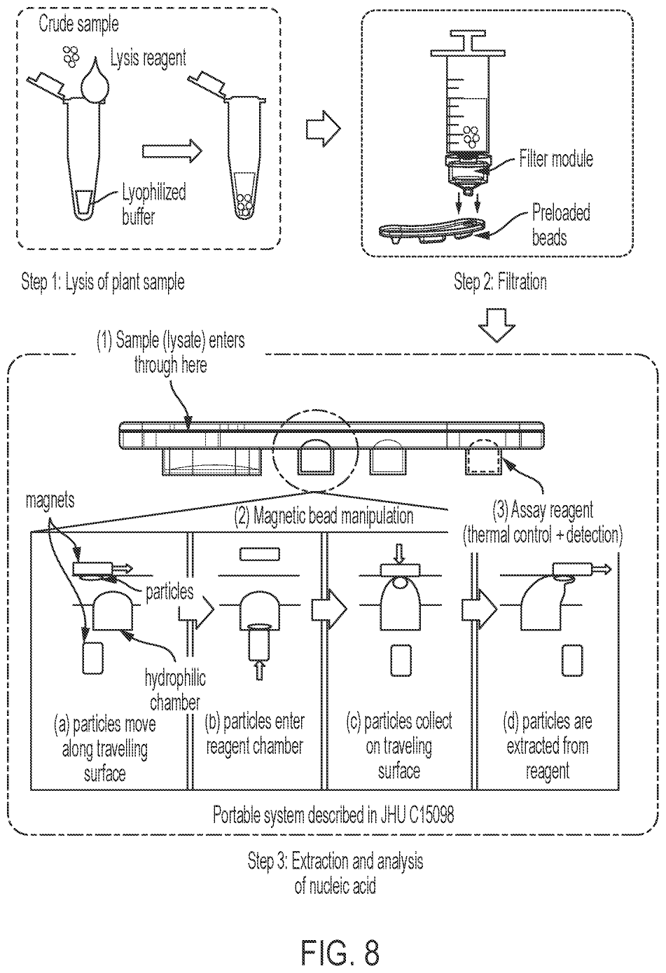

The overall genetic analysis of plant material according to an embodiment of the invention is more fully described in FIG. 8. Briefly, the process is composed of three steps: (1) lysis, (2) filtration for debris removal, and (3) nucleic acid extraction and analysis on a portable instrument. More specifically, the three steps include (1) first, in the process of lysis of plant sample, an acidic buffer reconstitutes lysis chemicals that are pre-lyophilized in a tube. Crude sample of interest is added to the tube. The whole tube is mixed and incubated to breakdown plant cell wall and release DNA. (2) Secondly, in the process of filtration for debris removal, the lysate is drawn into a syringe and pass through a filter module assembly using pressure-driven method. The outlet of filter is directly connected to the inlet of cartridge to be tested on portable magnetofluidic platform for further analysis. (3) The third step involves nucleic acid extraction and analysis on a portable instrument.

Interface Device for Connecting Lysate Preparation with Assay Platform

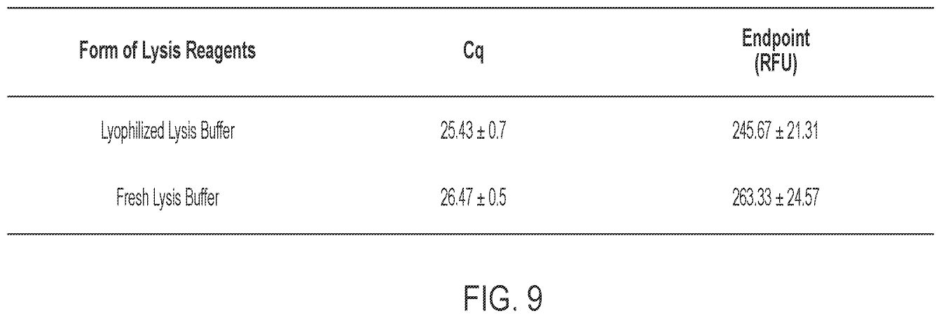

To simplify lysis process for end users, parts of chemicals involving in the lysis step are pre-lyophilized. This form of reagent preparation highly cuts down the number of pipetting times for users, as shown in FIG. 9.

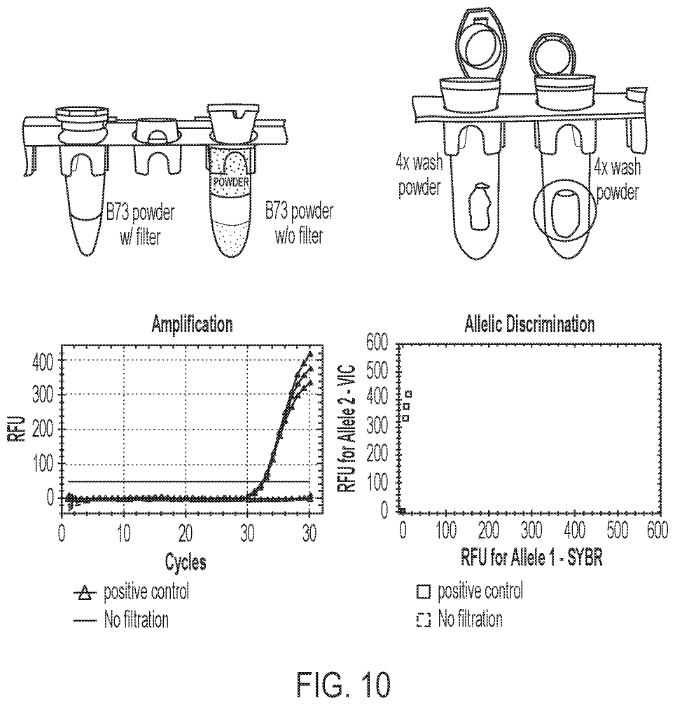

Plant cell lysate is commonly prepared via mechanical breakdown of plant material by methods including mortar and pestle and ultrasonication. Soft tissues may be processed directly via ultrasonication, while hard materials may require additional processing such as freezing and grinding. After plant cell tissues being processed to the form of powder or punch, several chemicals are added to breakdown cell wall and release DNA from plant cell sample. Samples prepared in this manner undergo further processing to precipitate out polysaccharides and polyphenols, which are known inhibitors of nucleic acid analysis assays. Unfortunately, that plant samples which are purified using solid-phase extraction without appropriate removal of the precipitants result in the carryover of particulates, which cause inhibition of genetic analysis assays, as shown in FIG. 10.

FIG. 10 is a collection of images and graphs showing the filtration-based removal of precipitants. The top left panel shows a photograph of B73 maize seed sample which has been filtered and not filtered. The top right panel shows magnetic particles exposed to sample. Sample with no precipitant (left) shows a clean magnetic particle cluster after washing, while the unfiltered sample (right) shows a bulky, contaminated particle cluster. The bottom panel show that lysate without filtration can't be amplified. Positive control is sample using filtration before bead extraction, no filtration sample is directly extracted using beads without pre-filtration.

Accordingly, what is needed in the art is an interface device that can connect a reservoir containing the plant lysate with an assay platform; at the same time, the device can provide the removal function of the precipitants in plant lysate when the lysate is transferred from the reservoir into the assay platform.

An embodiment of the invention addresses this by utilizing a filter module assembly, which removes particulates from the solution to enable real-time detection of DNA markers via probe-based real-time nucleic acid amplification testing assay. The device described herein can be used for integrating plant lysate input to the downstream assay platform.

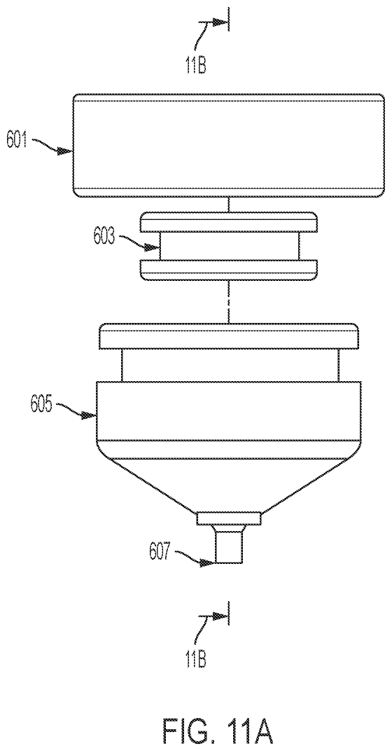

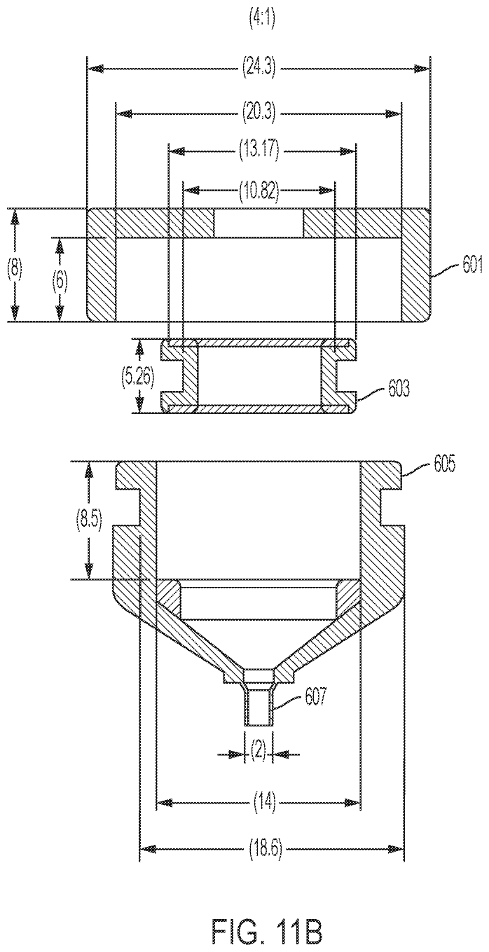

As shown in FIGS. 11A, 11B, 12A, 12B, and 13A-13D an embodiment of the filter module assembly described herein includes an upper portion 601 providing an attachable hub 602 to a syringe containing plant's chemical lysate, a middle layer 603 that can incorporate series of filter membranes (not shown), and a lower device portion 605 which the middle layer 603 is placed in and provide attachable hub 607 to the droplet magnetofluidic reagent scaffold system. The upper portion 601 provides an attachable hub to a syringe 602 by either a luer lock or a luer slip connection. The lower device portion 605 includes a barrel portion that provides the room for the placement of the middle layer portion 603 and an outward projection 607 which can be attached to a downstream analysis vial or to a microfluidic cartridge by either a luer slip or permanent connection during manufacturing process. When a microfluidic cartridge 609 is used for downstream analysis, the length of the outward projection portion 607 of the lower device portion 605 should exceed the thickness of the upper cartridge portion 611 of the microfluidic cartridge 609 for a certain distance to avoid the filtered plant sampled from contacting any hydrophobic layers in the microfluidic cartridge.

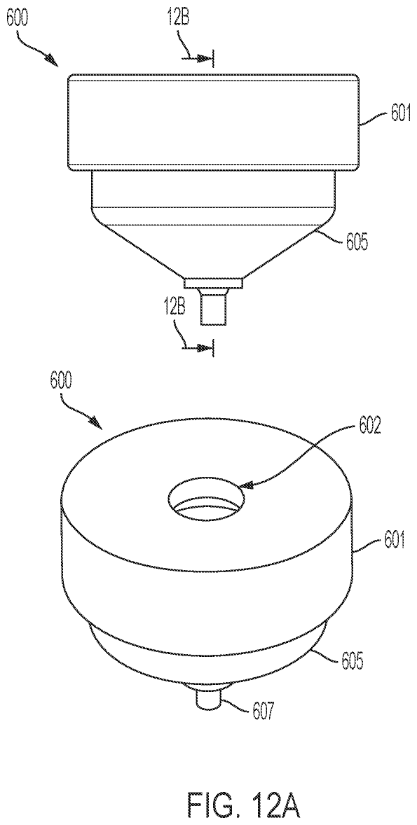

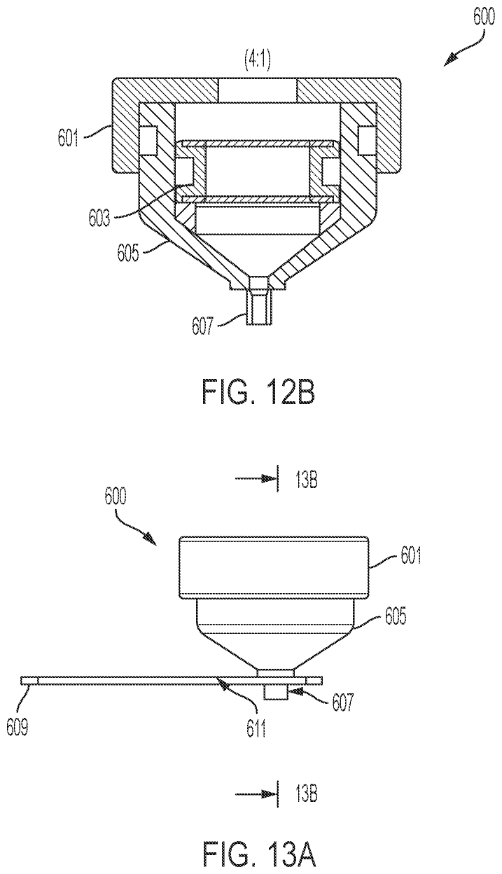

FIG. 11A is an exploded view of a filter module assembly whose upper part 601 can be attached to a syringe containing plant lysate (not shown) and whose lower part 605 can be attached to a microfluidic cartridge 609. The filter module assembly is composed of three parts: upper portion 601, middle layer 603, and lower portion 605. FIG. 11B is a crosssectional view of the assembly of FIG. 11A. All dimensions are displayed in millimeters (mm), and can be modified based on the input liquid volume. FIG. 12A is an assembled side view and perspective view of the filter module assembly 600 of FIGS. 11A and 11B. FIG. 12B is a cross-sectional view of the filter module assembly 600 of FIG. 12A.

In an embodiment according to the invention, the materials of the upper portion and lower device portion are surface modified to avoid the biomolecule of interest, for example nucleic acid, from sticking to the surface of these components. The upper portion and lower portion of this filter module assembly can be disposable or reused after thoroughly bleached to eliminate chances of contamination. The middle layer can include series of filter membrane to provide a removal function of cellular debris from plant lysate. The pore size and material of filter membrane depend upon the particular use, and the preferred material of filter membrane should be able to sustain high acidic solution and low static charge to prevent the loss of nucleic acid. In this particular use of genotyping using nucleic acid from plant lysate, pore size of filter should not be smaller than 2.0 μm to ensure the yield of nucleic acid after filtration. Furthermore, a second layer of filter membrane with pore size larger than 20.0 μm can be added to remove the larger cellular debris before lysate reaches the finer filter membrane. The material of the second filter layer can be nylon as an example.

The overall assembly of the filter module assembly 600 is depicted in FIGS. 12A and 12B. The dimension of the filter module assembly 600 is determined by the diameter of the filter membrane (not shown) in the middle layer 603. The larger volume of filtrate required to be collected, the larger diameter of the filter membrane and the wider of the filter module assembly should be. If one aims to collect up to 500 μL filtrate from the plant lysate, a diameter larger than 13 mm of filter membrane should be used. In some particular uses where less than 150 μL filtrate is aimed to be collected or the interface device is connected to a droplet magnetofluidic-assisted sample processing cartridge, the diameter of the filter membrane can be smaller than 13 mm. The diameter of the filter membrane and the width of interface device should not exceed sizes that lead to the situation such that the retention volume is too large for enough filtrate to be collected.

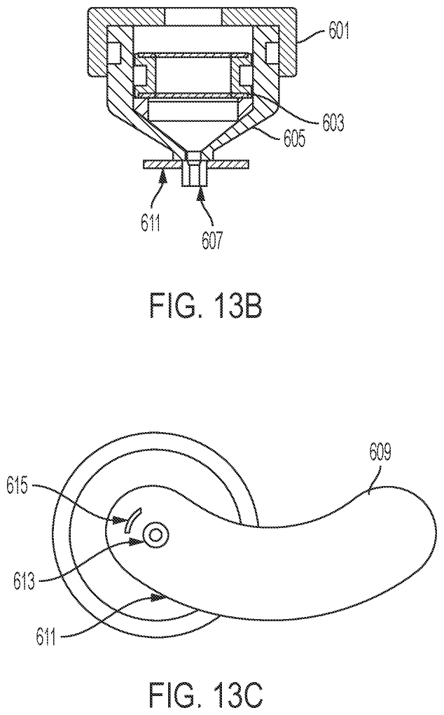

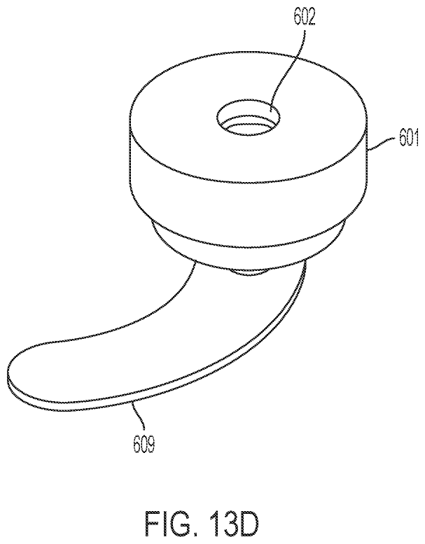

To use the filter module assembly 600, a syringe containing plant lysate is attached to the hub 602 of the upper portion 601 of the filter module assembly 600. The outward projection 607 of the lower portion can be either connected to a microfluidic cartridge 609 prior to the syringe attachment or after, as demonstrated in FIGS. 13A-13D. FIGS. 13A-13D are schematics showing integration of the filter module assembly 600 connecting with an upper portion 611 of a microfluidic cartridge 609 for downstream nucleic acid extraction and analysis. Specifically, FIG. 13A is an illustration showing a side view of the filter module assembly 600 connected to a microfluidic cartridge 609. FIG. 13B is a cross-sectional view of the filter module assembly 600 of FIG. 13A. FIGS. 13C and 13D are bottom and perspective views of the filter module assembly of FIG. 13A, respectively. In FIGS. 13A-13D, the outward projection portion 607 of the lower device portion 605 should exceed the thickness of upper cartridge portion 611. FIG. 13C is a bottom view and shows that the upper cartridge portion 611 has two openings; one opening 613 is the inlet for the insertion of outward projection 607 of filter module assembly, another opening 615 acts as a vent where excess air is pushed out.

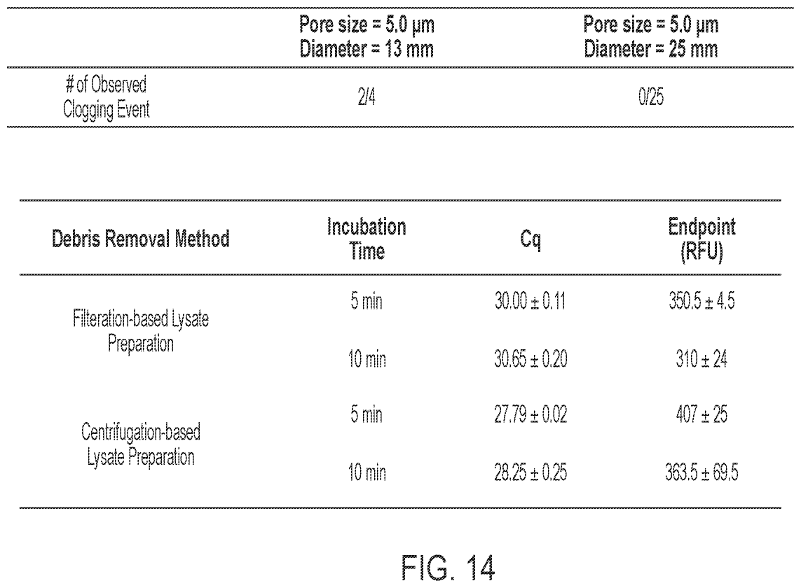

A syringe plunger is actuated by a user and this actuation activates the movement of plant lysate from the syringe into the filter module assembly. The particles larger than the pore size of the filter membrane in the lysate will be separated on top of the middle layer if the device includes a filter membrane. The clear filtrate medium is then passed through the filter module assembly into a sample well in the microfluidic cartridge and displaces any air occupying the sample well; the air escapes through another opening at the upper cartridge portion adjacent to the sample well. The filter module assembly should be able to sustain a pressure of up to about 180 PSI. The performance of filter module assembly was evaluated by comparing to a centrifugation-based lysate preparation process as shown in FIG. 14. FIG. 14 is a table disclosing the performance of filtration-based lysate preparation. The top panel shows that performance of the filtration method is generally agnostic to the pore size of filter used, for the pore size ranges 0.45 to 5 microns. The middle panel shows that a filter membrane with a larger diameter helps to reduce the occurrence rate of a clogging event during filtration for lysate up to 1 mL. The bottom panel shows that performance of filtration method is not affected by incubation time above 5 minutes.

Nucleic Acid Purification and Analysis

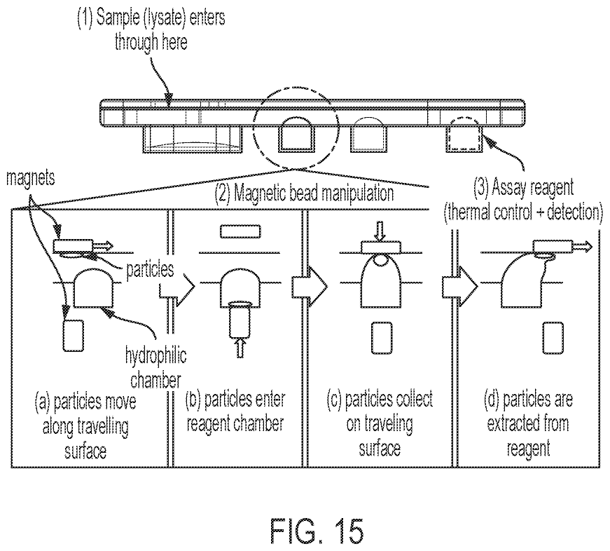

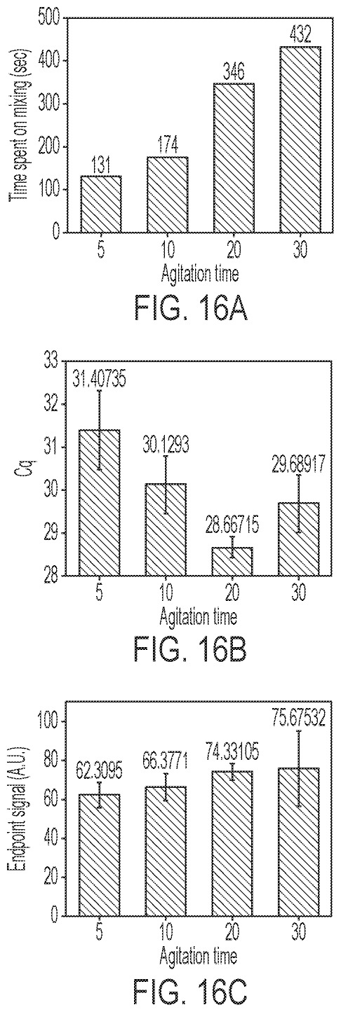

An embodiment of the invention utilizes a droplet magnetofluidic device as shown in FIG. 15 to facilitate the automated, portable method of nucleic acid purification and analysis from an unpurified liquid sample. The cartridge shown in FIG. 15 contains three wells preloaded with droplets of a magnetic beads, wash buffers, and a PCR reaction mix. The assay begins with injection of the filtered plant cell lysate from the outlet of the filter module assembly directly into the first well through a port in the cartridge. This first well contains the preloaded magnetic beads. Electrostatic forces cause binding between the magnetic beads and negatively-charged nucleic acids in solution. The agitation of beads in the first well, with the aid of a robotic arm with magnets, replaces the manually mixing of beads and nucleic acid in filtrate as demonstrated in FIGS. 16A-16C. After capturing of DNA from filtrate, the DNA-bound magnetic beads from the DNA binding buffer well to the wash buffer well and finally to a well for polymerase chain reaction (PCR).

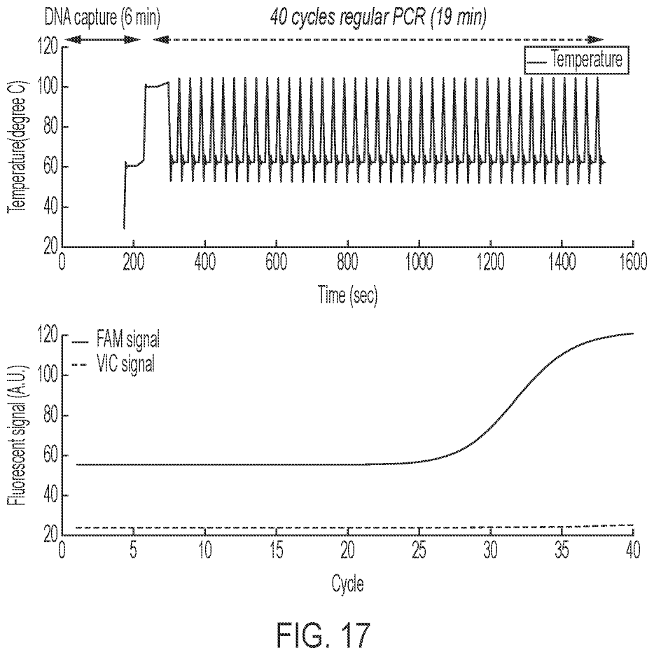

Transfer of the beads through a wash buffer well ensures that inhibitory components from the sample are desorbed from the beads while the pH maintains the positive charge on the beads for subsequent transfer of the captured nucleic acids into the PCR solution. PCR is inherently carried out in more basic solutions (pH=8-9), which neutralizes the magnetic bead charge for elution of sample nucleic acids into solution. The magnetic beads are then transferred out of the well, followed by thermal control and optical detection as required by the downstream analysis technique. The overall sample-to-result time is about 30 minutes as demonstrated in FIG. 17.

Allelic Discrimination Assay

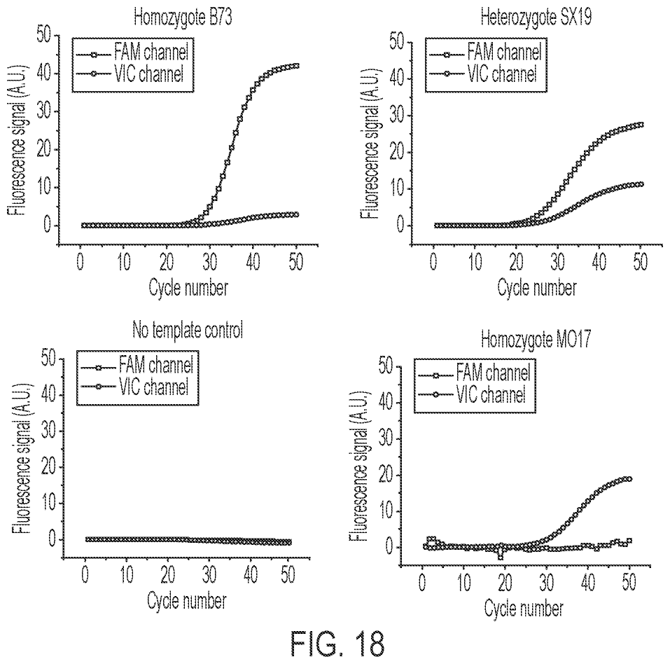

Next, three maize seed samples were tested using a hydrolysis probe PCR assay. The overall workflow for this assay paralleled the protocol illustrated in FIG. 7, using a combination of filtration-based lysate preparation and droplet magnetofluidic assay integration. As shown in FIG. 18, the results show that it is possible to accurately discriminate between samples that are positive homozygous or heterozygous for a biomarker of interest. FIG. 18 shows graphs showing the detection of hydrolysis probe markers using a PCR assay for maize samples MO17, SX19 and B73, using the complete laboratory-free workflow for plant lysate preparation, nucleic acid purification and analysis. The samples MO17 (homozygous VIC-positive for marker), SX19 (homozygous FAM-positive for marker) and SX19 (heterozygous for marker) all generate fluorescence signals as expected from their genotype.

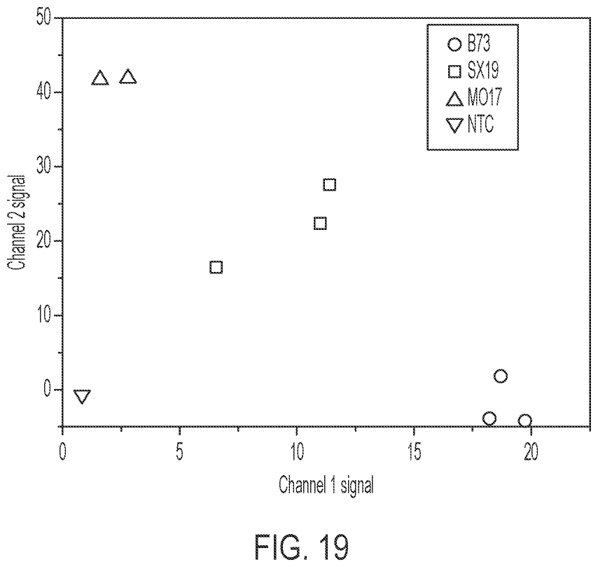

The lysate preparation protocol was characterized in isolation to verify that the nucleic acid samples obtained using the method are suitable for use in allelic discrimination. As shown in FIG. 19, the results were found to be in agreement with the conventional Hot Shot DNA extraction method for plant samples.

Biomarker Quantification Assay

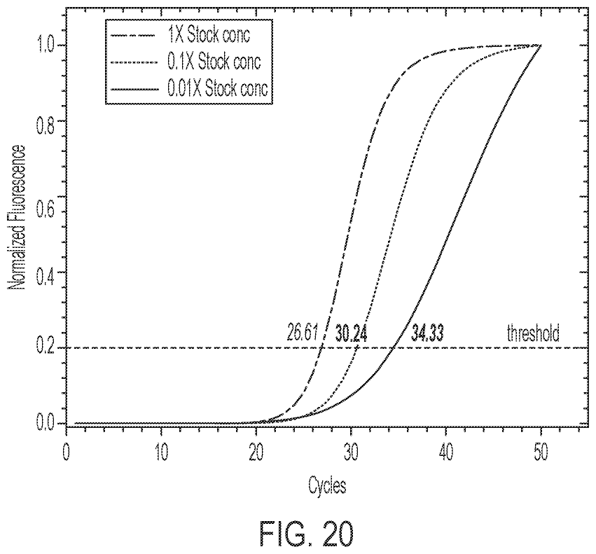

Next, the ability to quantify varying amount of nucleic acid targets was tested. Samples were prepared in ten-fold dilutions and tested directly on a magnetofluidic assay cartridge for hydrolysis probe PCR amplification. As shown in FIG. 20, the resulting signal shows a delay in amplification threshold cycle by 3-4 cycles per decade, which is in agreement with typical observation from a quantitative PCR assay. FIG. 20 is a graph showing evaluation of plant biomarker quantification capability using dilutions of DNA in the droplet magnetofluidic scaffold device, using genomic DNA extracted from MO17 maize line. Shift of cycle threshold around 3-4 cycles for each 1:10 dilution indicate the ability to detect changes in concentration of targeted biomarker using a quantitative PCR assay.

### Example 3

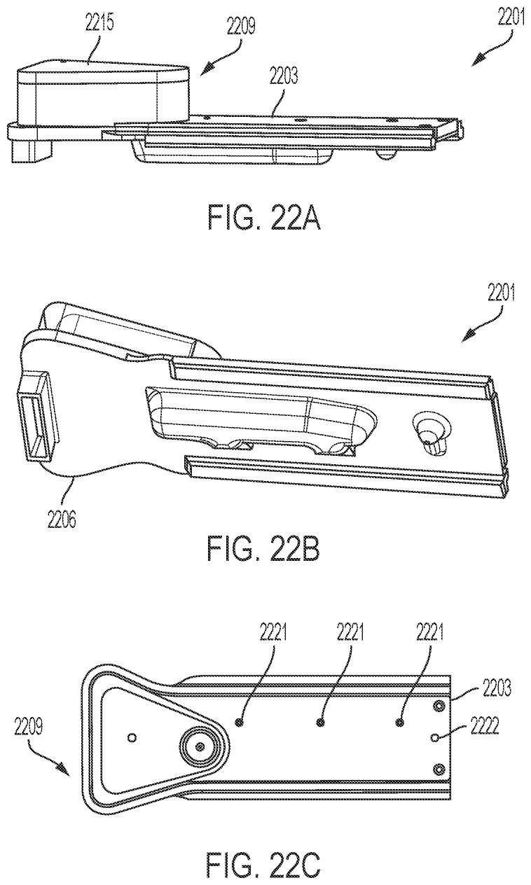

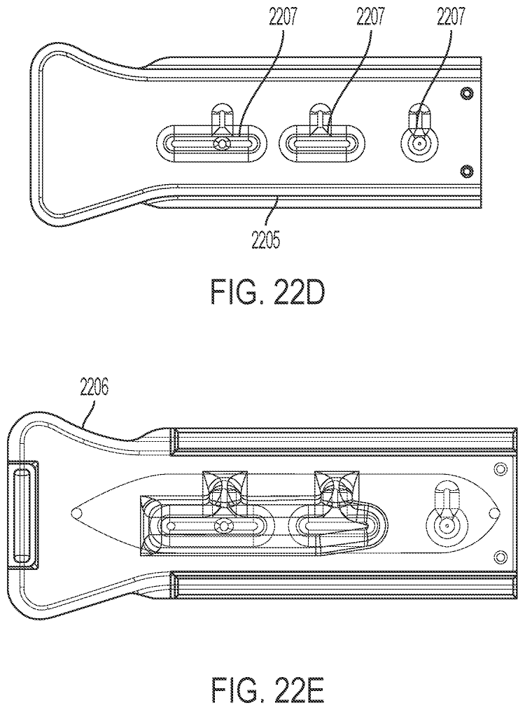

FIGS. 22A-22E are schematics of a device 2201 for assaying a biomolecule from a plant sample. In FIGS. 22A-22E, the device 2201 includes a top layer 2203, a bottom layer 2205 spaced apart from the top layer 2203 in a generally parallel orientation with respect to the top layer 2203, and a protective layer 2206 configured to attach to a bottom side of the bottom layer 2205. The bottom layer has one or more wells 2207 that protrude from the surface of the bottom layer 2205. The device also contains an integrated filter module 2209 for filtering the plant sample. The filter module has an inlet structure forming an inlet channel (not shown), and is configured to accept and secure a filter membrane (not shown). The filter module is configured to accept a microvolume aliquot of the plant sample. The filter module also includes a cap 2215 connected to the filter module 2209. The top layer 2203 also includes a plurality of open ports 2221 for the loading of one or more reagents, and an open port 2222 for loading silicone oil into the device prior to use. Specifically, FIG. 22A is a side view of the device. FIG. 22B is a bottom view of the device. FIG. 22C is a top view of the device. FIG. 22D is a bottom view of the bottom layer 2205 of the device. FIG. 22E is a transparent bottom view of the protective layer 2206. As seen in FIG. 22E, the protective layer 2206 is configured to accept one or more wells from the bottom layer 2205. The device is linear in configuration.

### Example 4

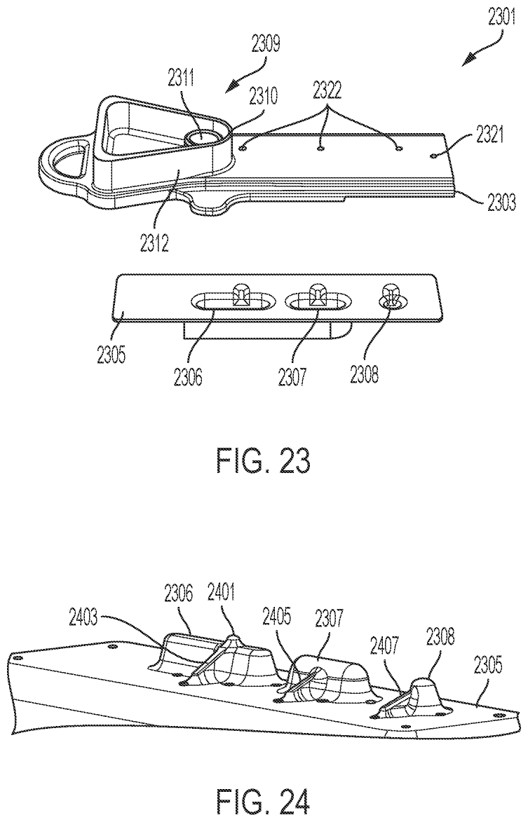

FIGS. 23-25 are schematics showing a device 2301 for assaying a biomolecule from a plant sample. The device 2301 includes a top layer 2303, and a bottom layer 2305 spaced apart from the top layer 2303 in a generally parallel orientation with respect to the top layer 2303. The bottom layer has one or more wells 2306, 2307, 2308 that protrude from the surface of the bottom layer 2305. The device also contains a permanently integrated filter module 2309 for filtering the plant sample. The filter module has an upper portion 2310 having an inlet structure forming an inlet channel 2311, and a bottom portion 2312 configured to accept and secure a filter membrane (not shown). The filter module 2309 is configured to accept a microvolume aliquot of the plant sample. The top layer 2303 also includes an open port 2321 for the loading of silicone oil into the device prior to use, and a series of reagent loading ports 2322 for loading one or more reagents into the plurality of wells. At least the first of the plurality of wells contains reagents for a biological assay. In some embodiments, this well also contains magnetic beads configured to bind to the biomolecule.

With respect to the specifications of the plurality of wells, the sidewalls are smooth and tapered, where bottom cross section is smaller than the top. The wells are in general square shaped for the sample 2306 and rinse well 2307 and circular for the assay well 2308. The assay well 2308 is configured to be operably connected to a PCR assay device. The long axis of the square is parallel to the direction of flow for magnetic beads. The well depth is limited so that magnets can move the beads on top exterior and bottom exterior of the wells.

The sample well 2306 is configured to hold a sample having a volume between 50 to 250 ul. As shown in FIG. 24, the bottom of the sample well 2306 may form a bead retaining structure 2401 that descends below the base of the sample well 2306 and secures the beads in position during storage and shipment. The side of the well may be slopped relative to a sample dispensing tip and to the orientation of the cartridge in the instrument. The sample well 2306 contains a side loading channel 2403 that has a grove leading to the bottom of the sample well 2306. The side loading channel has a ramped design to allow a reagent to flow from a corresponding reagent loading port 2322 to the bottom of the sample well.

The rinse well 2307 is configured to hold a sample having a volume between 50 to 200 ul. As shown in FIG. 24, the rinse well contains a side loading channel 2405 that has a grove leading to the bottom of the rinse well 2307. The side loading channel has a ramped design to allow a reagent to flow from a corresponding reagent loading port 2322 to the bottom of the rinse well.

The assay well 2308 is configured to hold a sample having a volume between 10 to 50 ul. As shown in FIG. 24, the assay well contains a side loading channel 2407 that has a grove leading to the bottom of the assay well 2308. The side loading channel has a ramped design to allow a reagent to flow from a corresponding reagent loading port 2322 to the bottom of the assay well.

In some assays, magnetic beads are pre-loaded into the device, such as on the filter matrix, in a nozzle after the filter matrix, or in the sample well 2306. In embodiments where the beads are pre-loaded into the device, the sample well 2306 includes an additional bead retaining structure 2401 that descends below the base of the loading well and secures the beads in that position during storage and shipment. An outlet channel (see for example structure 1823 in FIGS. 3B and 3C) may be optimally positioned to be above or proximal to the bead retaining well.

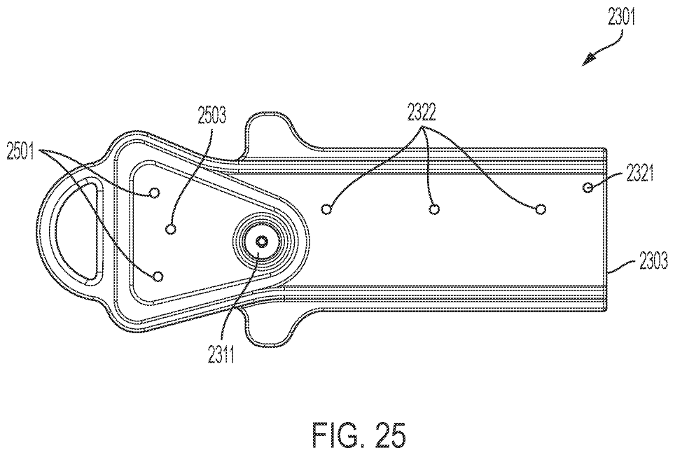

FIG. 25 shows a top view of the device of FIGS. 23 and 24. The device includes oil loading ports 2501 and an oil vent 2503. The reagent loading ports 2322 are positioned sch that they are off-set from the base of the one or more wells. This off-set configuration allows for a reagent to be loaded into the side loading channel 2403, 2405, 2407 of each of the respective wells.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. A device for assaying a biomolecule from a plant sample comprising:
a microfluidic cartridge for assaying a biomolecule from a plant sample, comprising:
a top layer; and
a bottom layer spaced apart from said top layer in a generally parallel orientation with respect to said top layer, said bottom layer defining a plurality of wells therein that protrude from a surface of said bottom layer; and
a filter module for filtering the plant sample, comprising a filter body defining:
an upper portion comprising an inlet structure forming an inlet channel; and
a bottom portion configured to accept and secure a filter membrane,
wherein said filter body is configured to accept a microvolume aliquot of said plant sample,
wherein said bottom structure comprises an outlet structure forming an outlet channel on an outlet side of said filter membrane, and
wherein at least one of said plurality of wells comprises an assay reagent solution.

2. The device of claim 1, wherein at least one of said plurality of wells contains a plurality of magnetic beads, and wherein said plurality of magnetic beads are configured to bind to said biomolecule.

3. The device of claim 1, wherein said outlet structure is configured to mechanically connect said bottom structure with said inlet of the top layer of the microfluidic cartridge.

4. (canceled)

5. The device of claim 1, wherein said filter module further comprises a cap structure comprising a plunger complementary to said inlet channel, such that when in use, said plunger occupies said inlet channel.

6.-10. (canceled)

11. The device of claim 1, further comprising a filter membrane disposed in said bottom portion, wherein said filter membrane comprises an average ensemble pore size of up to 20 micrometers in diameter.

12. (canceled)

13. The device of claim 1, wherein the filter body is a multi-component assembly comprising:
a filter module for filtering the plant sample and configured to mechanically connect to the microfluidic cartridge, the filter module comprising:
an upper portion comprising an inlet structure forming an inlet channel;
a middle layer configured to accept and secure a filter membrane; and
a bottom portion configured to accept said middle layer,
wherein said upper portion and said bottom portion are configured to couple with one another to form a assembly such that said middle layer is disposed within said fluid-tight assembly during use,
wherein said fluid-tight assembly is configured to accept a microvolume aliquot of said plant sample,
wherein said bottom portion comprises an outlet structure forming an outlet channel on an outlet side of said middle layer, and
wherein said outlet structure is configured to mechanically connect said bottom portion with said inlet of the top layer of the microfluidic cartridge.

14. The device of claim 13, further comprising a filter membrane disposed in said middle layer, wherein said filter membrane comprises an average ensemble pore size of up to 20 micrometers in diameter.

15. (canceled)

16. The device of claim 13, further comprising a second filter membrane disposed in said middle layer, such that said second filter membrane is in an anterior orientation during use with respect to said filter membrane.

17. The device of claim 16, wherein said second filter membrane comprises an average ensemble pore size of up to 20 micrometers in diameter.

18.-19. (canceled)

20. The device of claim 1, wherein at least one of said plurality of wells is a sample well configured to receive the plant sample therein, said sample well further comprises a bead retaining structure configured to descend below a base portion of said sample loading well.

21. The device of claim 1, wherein at least one of said plurality of wells is an assay well, said assay well configured to operably engage with to a thermocycling element of an assay device.

22.-25. (canceled)

26. The device of claim 1, wherein said device further comprises an adapter configured to mechanically connect said filter module to said microfluidic cartridge.

27.-29. (canceled)

30. A filter module for filtering a plant sample, comprising a fluid-tight filter body defining:
an upper portion comprising an inlet structure forming an inlet channel; and
a bottom portion configured to accept and secure a filter membrane,
wherein said fluid-tight filter body is configured to accept a microvolume aliquot of said plant sample,
wherein said bottom portion comprises an outlet structure forming an outlet channel on an outlet side of said filter membrane, and
wherein said outlet structure is configured to mechanically connect said bottom portion with a microfluidic cartridge.

31. The filter module of claim 30, further comprising a filter membrane disposed in said bottom portion, wherein said filter membrane comprises an average ensemble pore size of up to 2 micrometers in diameter.

32.-33. (canceled)

34. The filter module of claim 30, wherein the fluid-tight filter body is a multi-component assembly comprising:
an upper portion comprising an inlet structure forming an inlet channel;
a middle layer configured to accept and secure a filter membrane; and
a bottom portion configured to accept said middle layer,
wherein said upper portion and said bottom portion are configured to couple with one another to form a fluid-tight assembly such that said middle layer is disposed within said fluid-tight assembly during use,
wherein said fluid-tight assembly is configured to accept a microvolume aliquot of said plant sample,
wherein said bottom portion comprises an outlet structure forming an outlet channel on an outlet side of said middle layer, and
wherein said outlet structure is configured to mechanically connect said bottom portion with a microfluidic cartridge.

35. The filter module of claim 34, further comprising a filter membrane disposed in said middle layer, wherein said filter membrane comprises an average ensemble pore size of up to 2 micrometers in diameter.

36. The filter module of claim 35, further comprising a second filter membrane disposed in said middle layer, such that said second filter membrane is in an anterior orientation during use with respect to said filter membrane.

37. The filter module of claim 36, wherein said second filter membrane comprises an average ensemble pore size of up to 20 micrometers in diameter.

38.-45. (canceled)

46. A method of detecting a biomolecule in a plant sample, comprising:
preparing a lysate comprising said plant sample by contacting said plant sample with a lysis buffer;
filtering a microvolume aliquot of said lysate using a filter module;
loading the filtered plant sample into a sample well of a microfluidic cartridge;
amplifying the biomolecule; and
detecting the biomolecule,
wherein said preparing said lysate and said filtering said microvolume aliquot of said lysate are done at an ambient temperature.

47. The method of claim 46, wherein the filter module comprises:
an upper portion comprising an inlet structure forming an inlet channel; and
a bottom portion configured to accept and secure a filter membrane,
wherein said filter assembly is configured to accept a microvolume aliquot of said plant sample in said inlet channel,
wherein said bottom portion comprises an outlet structure forming an outlet channel on an outlet side of said filter membrane, and
wherein said outlet structure has a length so as to extend into a well in said bottom layer without reaching a bottom of said well.

48.-50. (canceled)
